SEQUENCE LISTING (94 sequences)

SEQ ID NO: 1 - protein, 453 aa, synthetic construct
  REGION 1..453: note = FS22-172-003-AA/FS28-256-271 mAb2: Heavy chain AA (without LALA)
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 2 - protein, 453 aa, synthetic construct
  REGION 1..453: note = FS22-172-003-AA/FS28-256-271 mAb2: Heavy chain AA (with LALA)
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 3 - protein, 119 aa, synthetic construct
  REGION 1..119: note = FS22-172-003-AA/FS28-256-271 mAb2: VH domain AA
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSS

SEQ ID NO: 4 - protein, 7 aa, synthetic construct
  REGION 1..7: note = FS22-172-003-AA/FS28-256-271 mAb2: LCDR1 (AA) (IMGT)
QSVSSSY

SEQ ID NO: 5 - protein, 5 aa, synthetic construct
  REGION 1..5: note = HCDR1 (AA) (Kabat)
HTYMS

SEQ ID NO: 6 - protein, 8 aa, synthetic construct
  REGION 1..8: note = HCDR2 (AA) (IMGT)
ISPTYSTT

SEQ ID NO: 7 - protein, 17 aa, synthetic construct
  REGION 1..17: note = HCDR2 (AA) Kabat)
AISPTYSTTNYADSVKG

SEQ ID NO: 8 - protein, 12 aa, synthetic construct
  REGION 1..12: note = HCDR3 (AA) (IMGT)
ARYNAYHAALDY

SEQ ID NO: 9 - protein, 10 aa, synthetic construct
  REGION 1..10: note = HCDR3 (AA) (Kabat)
YNAYHAALDY

SEQ ID NO: 10 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQTVPYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQTVPYPYTFGQGTKVEIK

SEQ ID NO: 12 - protein, 7 aa, synthetic construct
  REGION 1..7: note = LCDR1 (AA) (IMGT)
QSVSSSY

SEQ ID NO: 13 - protein, 12 aa, synthetic construct
  REGION 1..12: note = LCDR1 (AA) (Kabat)
RASQSVSSSYLA

SEQ ID NO: 14 - protein, 4 aa, synthetic construct
  REGION 1..4: note = LCDR2 (AA) (IMGT)
GASG

SEQ ID NO: 15 - protein, 7 aa, synthetic construct
  REGION 1..7: note = LCDR2 (AA) (Kabat)
GASSRAT

SEQ ID NO: 16 - protein, 9 aa, synthetic construct
  REGION 1..9: note = LCDR3 (AA) (IMGT)
QQTVPYPYT

SEQ ID NO: 17 - protein, 9 aa, synthetic construct
  REGION 1..9: note = LCDR3 (AA) (Kabat)
QQTVPYPYT

SEQ ID NO: 18 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 19 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSS

SEQ ID NO: 20 - protein, 8 aa, synthetic construct
  REGION 1..8: note = HCDR1 (AA) (IMGT)
GFTLSYSS

SEQ ID NO: 21 - protein, 5 aa, synthetic construct
  REGION 1..5: note = HCDR1 (AA) (Kabat)
YSSMS

SEQ ID NO: 22 - protein, 8 aa, synthetic construct
  REGION 1..8: note = HCDR2 (AA) (IMGT)
ITPSTGYT

SEQ ID NO: 23 - protein, 17 aa, synthetic construct
  REGION 1..17: note = HCDR2 (AA) Kabat)
FITPSTGYTHYADSVKG

SEQ ID NO: 24 - protein, 9 aa, synthetic construct
  REGION 1..9: note = HCDR3 (AA) (IMGT)
ARRALLFDY

SEQ ID NO: 25 - protein, 7 aa, synthetic construct
  REGION 1..7: note = HCDR3 (AA) (Kabat)
RALLFDY

SEQ ID NO: 26 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQASSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQASSYPLTFGQGTKVEIK

SEQ ID NO: 28 - protein, 9 aa, synthetic construct
  REGION 1..9: note = LCDR3 (AA) (IMGT)
QQASSYPLT

SEQ ID NO: 29 - protein, 453 aa, synthetic construct
  REGION 1..453: note = FS22-172-003-AA/FS28-256-021 mAb2: Heavy chain AA (without LALA)
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 30 - protein, 453 aa, synthetic construct
  REGION 1..453: note = Heavy chain AA (with LALA)
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 31 - protein, 17 aa, synthetic construct
  REGION 1..17: note = HCDR2 (AA) Kabat)
NISPTYSTTNYADSVKG

SEQ ID NO: 32 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQHNQYPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQHNQYPNTFGQGTKVEIK

SEQ ID NO: 34 - protein, 9 aa, synthetic construct
  REGION 1..9: note = LCDR3 (AA) (IMGT) and (Kabat)
QQHNQYPNT

SEQ ID NO: 35 - protein, 453 aa, synthetic construct; identical to SEQ ID NO: 29
  REGION 1..453: note = FS22-172-003-AA/FS28-256-012 mAb2: Heavy chain AA (without LALA)

SEQ ID NO: 36 - protein, 453 aa, synthetic construct; identical to SEQ ID NO: 30
  REGION 1..453: note = Heavy chain AA (with LALA)

SEQ ID NO: 37 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQSYYYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQSYYYPITFGQGTKVEIK

SEQ ID NO: 39 - protein, 9 aa, synthetic construct
  REGION 1..9: note = LCDR3 (AA) (IMGT) and (Kabat)
QQSYYYPIT

SEQ ID NO: 40 - protein, 453 aa, synthetic construct
  REGION 1..453: note = FS22-172-003-AA/FS28-256-023 mAb2: Heavy chain AA (without LALA)
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 41 - protein, 354 aa, synthetic construct
  REGION 1..354: note = Heavy chain AA (with LALA)
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLP

SEQ ID NO: 42 - protein, 119 aa, synthetic construct
  REGION 1..119: note = VH domain AA
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSS

SEQ ID NO: 43 - protein, 8 aa, synthetic construct
  REGION 1..8: note = HCDR1 (AA) (IMGT)
GFTFTQTY

SEQ ID NO: 44 - protein, 5 aa, synthetic construct
  REGION 1..5: note = HCDR1 (AA) (Kabat)
QTYMS

SEQ ID NO: 45 - protein, 12 aa, synthetic construct
  REGION 1..12: note = HCDR3 (AA) (IMGT)
ARYNAYQIGLDY

SEQ ID NO: 46 - protein, 10 aa, synthetic construct
  REGION 1..10: note = HCDR3 (AA) (Kabat)
YNAYQIGLDY

SEQ ID NO: 47 - protein, 215 aa, synthetic construct
  REGION 1..215: note = FS22-172-003-AA/FS28-256-024 mAb2:Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQALGYPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQALGYPHTFGQGTKVEIK

SEQ ID NO: 49 - protein, 9 aa, synthetic construct
  REGION 1..9: note = LCDR3 (AA) (IMGT) and (Kabat)
QQALGYPHT

SEQ ID NO: 50 - protein, 453 aa, synthetic construct
  REGION 1..453: note = FS22-172-003-AA/FS28-256-001 mAb2: Heavy chain AA (without LALA)
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 51 - protein, 453 aa, synthetic construct
  REGION 1..453: note = Heavy chain AA (with LALA)
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 52 - protein, 119 aa, synthetic construct
  REGION 1..119: note = VH domain AA
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSS

SEQ ID NO: 53 - protein, 8 aa, synthetic construct
  REGION 1..8: note = HCDR1 (AA) (IMGT)
GFTFTETY

SEQ ID NO: 54 - protein, 5 aa, synthetic construct
  REGION 1..5: note = HCDR1 (AA) (Kabat)
ETYMS

SEQ ID NO: 55 - protein, 12 aa, synthetic construct
  REGION 1..12: note = HCDR3 (AA) (IMGT)
ARYNSYQGGLDY

SEQ ID NO: 56 - protein, 10 aa, synthetic construct
  REGION 1..10: note = HCDR3 (AA) (Kabat)
YNSYQGGLDY

SEQ ID NO: 57 - protein, 453 aa, synthetic construct
  REGION 1..453: note = FS22-172-003-AA/FS28-256-014 mAb2: Heavy chain AA (without LALA)
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 58 - protein, 453 aa, synthetic construct
  REGION 1..453: note = Heavy chain AA (with LALA)
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 59 - protein, 119 aa, synthetic construct
  REGION 1..119: note = VH domain AA
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSS

SEQ ID NO: 60 - protein, 8 aa, synthetic construct
  REGION 1..8: note = HCDR1 (AA) (IMGT)
GFTFTDTY

SEQ ID NO: 61 - protein, 5 aa, synthetic construct
  REGION 1..5: note = HCDR1 (AA) (Kabat)
DTYMS

SEQ ID NO: 62 - protein, 12 aa, synthetic construct
  REGION 1..12: note = HCDR3 (AA) (IMGT)
ARYNAYAAGLDY

SEQ ID NO: 63 - protein, 10 aa, synthetic construct
  REGION 1..10: note = HCDR3 (AA) (Kabat)
YNAYAAGLDY

SEQ ID NO: 64 - protein, 453 aa, synthetic construct
  REGION 1..453: note = of FS22-172-003-AA/FS28-256 mAb2: Heavy chain AA (without LALA)
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 65 - protein, 453 aa, synthetic construct
  REGION 1..453: note = Heavy chain AA (with LALA)
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 66 - protein, 118 aa, synthetic construct
  REGION 1..118: note = VH domain AA
VQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSS

SEQ ID NO: 67 - protein, 8 aa, synthetic construct
  REGION 1..8: note = HCDR1 (AA) (IMGT)
GFTFTNTY

SEQ ID NO: 68 - protein, 5 aa, synthetic construct
  REGION 1..5: note = HCDR1 (AA) (Kabat)
NTYMS

SEQ ID NO: 69 - protein, 450 aa, synthetic construct
  REGION 1..450: note = FS22-172-003-AA/FS28-024-051 mAb2: Heavy chain AA (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 70 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 71 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSS

SEQ ID NO: 72 - protein, 9 aa, synthetic construct
  REGION 1..9: note = HCDR3 (AA) (IMGT)
ARRALIFDY

SEQ ID NO: 73 - protein, 7 aa, synthetic construct
  REGION 1..7: note = HCDR3 (AA) (Kabat)
RALIFDY

SEQ ID NO: 74 - protein, 450 aa, synthetic construct
  REGION 1..450: note = FS22-172-003-AA/FS28-024-053 mAb2: Heavy chain AA (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 75 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 76 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSS

SEQ ID NO: 77 - protein, 9 aa, synthetic construct
  REGION 1..9: note = HCDR3 (AA) (IMGT)
ARRALVFDY

SEQ ID NO: 78 - protein, 7 aa, synthetic construct
  REGION 1..7: note = HCDR3 (AA) (Kabat)
RALVFDY

SEQ ID NO: 79 - protein, 450 aa, synthetic construct
  REGION 1..450: note = FS22-172-003-AA/FS28-024 mAb2: Heavy chain AA (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 80 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 81 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSS

SEQ ID NO: 82 - protein, 9 aa, synthetic construct
  REGION 1..9: note = HCDR3 (AA) (IMGT)
ARRALTFDY

SEQ ID NO: 83 - protein, 7 aa, synthetic construct
  REGION 1..7: note = HCDR3 (AA) (Kabat)
RALTFDY

SEQ ID NO: 84 - protein, 448 aa, synthetic construct
  REGION 1..448: note = FS122m (surrogate anti-mMSLN mCD137 Fcab G1-AA): Heavy chain AA (with LALA)
EVQLLESGGGLVQPGGSLRLSCAASGFTFSTYFMVWVRQAPGKGLEWVSMISPKSSNTYYADSVKGRFTISRDNSKNTLYLQMNSLRAEDTAVYYCARYHISPRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEPYWSYVSLTCLVKGFYPSDIAVEWESNGQPENNYKTTPPVLDSDGSFFLYSKLTVMNYRWELGNVFSCSVMHEALHNHYTQKSLSLSPGK

SEQ ID NO: 85 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQPFPFSFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 111 aa, synthetic construct
  REGION 1..111: note = CH3 AB and EF structural loops of all FS22-172-003 Fcab-containing mAb2 clones and the FS22-172-003 Fcab: CH3 domain
GQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 87 - protein, 7 aa, synthetic construct
  REGION 1..7: note = AB Loop
PYIIPPY

SEQ ID NO: 88 - protein, 7 aa, synthetic construct
  REGION 1..7: note = EF Loop
GADRWLE

SEQ ID NO: 89 - protein, 9 aa, synthetic construct
  REGION 1..9: note = CD Loop
SNGQPENNY

SEQ ID NO: 90 - protein, 110 aa, synthetic construct
  REGION 1..110: note = CH2 domain containing LALA mutation, PA mutation or LALA-PA mutation: CH2 (LALA)
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 91 - protein, 110 aa, synthetic construct
  REGION 1..110: note = CH2 (PA)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 92 - protein, 110 aa, synthetic construct
  REGION 1..110: note = CH2 (LALA-PA)
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 93 - protein, 447 aa, synthetic construct
  REGION 1..447: note = pembrolizumab: Heavy chain AA
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 218 aa, synthetic construct
  REGION 1..218: note = Light chain AA
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0356965 A1
(43) Pub. Date: Nov. 12, 2026

(54) COMBINATION CANCER TREATMENT COMPRISING AN ANTI-MSLN/CD137 ANTIBODY AND PD-1/PD-L1 INHIBITOR

(71) Applicant: MERCK PATENT GMBH, Darmstadt (DE)

(72) Inventor: Chunxiao XU, Billerica, MA (US)

(73) Assignee: MERCK PATENT GMBH, Darmstadt (DE)

(21) Appl. No.: 19/106,452

(22) PCT Filed: Aug. 23, 2023
(86) PCT No.: PCT/EP2023/073074

§ 371 Date: Feb. 25, 2025

Related U.S. Application Data
(60) Provisional application No. 63/401,290, filed on Aug. 26, 2022.

(51) Int. Cl. C07K 16/28; A61K 39/00; A61P 35/00; C07K 16/32

(52) U.S. Cl. CPC C07K 16/2878; A61P 35/00; C07K 16/2818; C07K 16/32; A61K 2039/507; C07K 2317/31; C07K 2317/526; C07K 2317/565; C07K 2317/76

(57) ABSTRACT

The application relates to the use of an antibody molecule that binds to MSLN and CD137 and a PD-1/PD-L1 inhibitor in the treatment of cancer in a patient.

### FIELD OF THE INVENTION

The present invention relates to the use of a bispecific antibody molecule that binds to MSLN and CD137 and a PD-1/PD-L1 inhibitor in the treatment of cancer in a patient.

### BACKGROUND

Antagonist antibodies targeting immune checkpoint coinhibitory receptors can reverse immune resistance of some tumors, but the majority of patients do not respond to treatment or eventually experience resistance¹. Costimulatory pathways, such as the CD137/4-1BB pathway, are also important in driving productive anti-cancer immunity, with strong genetic evidence supporting their role in mediating anti-cancer immune responses²⁻⁶. Therefore, a growing number of studies aim to modify signals through the use of agonistic antibodies targeting costimulatory molecules to boost anti-tumor T cell responses.

CD137 (also known as 4-1BB or TNFRSF9) is an inducible T cell surface receptor belonging to the tumor necrosis factor receptor (TNFR) superfamily, which activates diverse cellular functions, including production of type 1 interferons and modulation of antigen-activated T cell survival⁷. CD137 is expressed on the surface of activated CD4⁺ and CD8⁺ T cells, monocytes, and B lymphocytes. The expression of CD137 can be induced via T cell receptor (TCR) stimulation⁸, which is termed “signal 1” (TCR/CD3/MHC interaction between human T cell and target cell). Activation of the CD137 pathway promotes T cell differentiation and survival⁹⁻¹¹, provides strong protection against activation-induced T cell death, and increases cytotoxicity¹²⁻¹⁴.

The efficacy of anti-CD137 therapy has been demonstrated in multiple non-clinical tumor models¹⁵⁻¹⁹ Anti-CD137 agonistic antibodies have been shown to induce effector molecule release from CD8⁺ T cells, increase proliferation, and prevent cytotoxic T lymphocyte (CTL) anergy, thereby breaking T cell tolerance towards tumor antigens²⁰ and increasing persistence of tumor-specific T cells²¹. Based on promising non-clinical anti-tumor effects, two 1^{st} generation CD137 agonists, utomilumab (PF-05082566) and urelumab (BMS-663513), have been developed and investigated clinically. Clinical studies of both utomilumab and urelumab monotherapy were suspended, however, due to low efficacy of utomilumab and hepatotoxicity of urelumab^{22,23}. Further structure analysis indicated that these outcomes were mediated by a recognized epitope on CD137 and by Fc gamma receptor (FcγR) ligand-dependent clustering²⁴.

To overcome either low anti-tumor efficacy or hepatotoxicity mediated by FcγR ligand-dependent clustering of the 1^{st} generation CD137 agonists, strategies that deliver CD137 agonists to the tumor site are required to reduce systemic toxicities while allowing for clinical administration²⁵. These 2^{nd} generation CD137 agonists are either monospecific antibodies claiming to bind CD137 epitopes that are not associated with liver toxicity or are CD137/tumor associated antigen (TAA) bispecific antibodies that are targeted to the tumor microenvironment (TME), do not bind FcγRs, and are linked to antibodies targeting tumor antigens or tumor tissue^{16,26,27}.

Mesothelin (MSLN) is a 40 kD membrane-bound protein that is overexpressed in various cancers, including mesothelioma, ovarian cancer, lung cancer, and pancreatic cancer²⁸⁻³⁹. The limited expression of MSLN on normal human tissues and its high expression in many common cancers make it an attractive candidate for cancer therapy. Several agents are in various stages of development to treat patients with MSLN-expressing tumors, including a monoclonal antibody, immunotoxin, tumor vaccine, and an antibody-drug conjugate⁴⁰.

M9657 (FS22-172-003-AA/FS28-256-271 of WO 2020/011976) is a first-in-class, tumor-targeted conditional agonist antibody developed to enhance anti-tumor immune responses in the TME. The bispecific antibody M9657 was engineered in a tetravalent bispecific antibody (mAb²) format, with the Fab portion binding the tumor antigen MSLN and a modified CH3 domain binding CD137. M9657 has a human IgG1-LALA backbone, which does not bind to Fcγ receptors but retains FcRn binding and IgG-like pharmacokinetics (PK). High expression of MSLN on tumor cells increases antibody binding to tumor cells, crosslinking of the antibody molecules, and interaction of the antibody molecules with the CD137 trimer, consequently increasing CD137 agonism. Therefore, the clustered M9657 may function as a bridge to link the CD137 trimer and tumor cells. As M9567 promotes CD137 activation signaling within the TME, which avoids systemic immune activation, it is expected that M9657 will provide advantages over monospecific CD137 antibodies. In preclinical studies, M9657 displayed MSLN target-dependent and dose-dependent anti-tumor immunity.

Monoclonal antibodies that block the interaction of programmed cell death protein 1 (PD-1) to its ligand programmed death-ligand 1 (PD-L1), such as an anti-PD-1/PD-L1 antibody, can enhance the immune response against cancer and are promising immune checkpoint antagonists. Currently, the FDA has approved 8 checkpoint inhibitors, including six anti-PD-1/PD-L1, one anti-CTLA-4 and one LAG3 monoclonal antibodies, for the treatment of more than a dozen major cancer types^{41,42}. The successful application of anti-PD-1/PD-L1 monoclonal antibodies in various clinical trials has showcased their remarkable potential in cancer immunotherapy. However, the clinical results were not always satisfactory, often showing large individual differences between patients, and with typically only a small number of patients responding to the treatment.

Thus, combination therapy has become a new research focus in the development of PD-1/PD-L1 blockade-based therapy. Anti-PD-1/PD-L1 antibodies and CD137 agonists have different and complementary mechanisms of action in cancer immunotherapy, specifically anti-PD-1/PD-L1 antibodies can abrogate negative signals that diminish T cell activation during the priming process or inhibit effector T cell activity at the tumor site, while CD137 agonists boost T cell activation via antigen presentation and cytokine secretion. The combination of PD-1/PD-L1 blockade with CD137 agonists has been shown to lead to improved T cell activation^{43,44}.

### STATEMENTS OF THE INVENTION

As explained in the background section, the combination of PD-1/PD-L1 inhibitors and CD137 agonists has been shown to improve T cell activation. However, the effect of such a combination in the treatment of cancer patients would not be limited to the tumor microenvironment, which raises concerns regarding the efficacy and specificity of such a treatment. Development of CD137 agonist molecules as anti-cancer therapies has also been held back due to concerns with regards to liver inflammation and clinical efficacy while anti-PD-1/PD-L1 monoclonal antibody therapies have shown large differences in outcomes between patients.

The present inventors recognized that there is a need for enhancing target-specific T cell activation and anti-tumor activity and that this could be achieved by combining MSLN expression-dependent CD137 co-stimulation with PD-1/PD-L1 inhibition. Surprisingly, the present inventors were able to show that the combination of an antibody molecule that binds MSLN and CD137 and a PD-1/PD-L1 inhibitor resulted in a greater anti-tumor effect in mouse tumor models than the combined anti-tumor effect observed when mice were treated with either the antibody molecule that binds MSLN and CD137, or the PD-1/PD-L1 inhibitor, alone. In other words, the anti-tumor effect of the combination treatment was not just additive but synergistic. This was unexpected. The effect achieved by a combination of two agents is synergistic if the effect is greater than the total of the individual effects of the two agents combined⁴⁵. Accordingly, the present inventors found that the combination of an antibody molecule that binds MSLN and CD137 and a PD-1/PD-L1 inhibitor increased the anti-tumor effect in mouse tumor models in a synergistic manner. A similar synergistic anti-tumor effect is expected when human patients are treated with a combination of an antibody molecule that binds MSLN and CD137 and a PD-1/PD-L1 inhibitor.

Due to the lack of cross-reactivity between M9657 (SEQ ID NO: 2 and SEQ ID NO: 10) and mouse MSLN and CD137 proteins, anti-mMSLN-mCD137-huIgG1-LALA (FS122m) (SEQ ID NO: 84 and SEQ ID NO: 85) was developed, a surrogate antibody of M9657 for the in vivo studies in mouse tumor models. As with M9657, FS122m was engineered in a tetravalent bispecific antibody (mAb²) format with the Fab portion targeted to bind to mouse MSLN and a modified CH3 domain targeted to mouse CD137. FS122m has a human IgG1 backbone with LALA mutations to abrogate binding to Fcγ receptors. The binding affinity of FS122m for mouse MSLN and mouse CD137 is similar to the binding affinity of M9657 for human MSLN and human CD137.

As already summarized above, the present inventors showed that the combination of FS122m and anti-mPD-1 was capable of retarding tumor growth or reducing tumor volume in E0771, JC and Eph4-1424 mouse tumor models to a greater extent than the combined tumor growth retardation or tumor volume reduction observed when mice were treated with either FS122m or anti-mPD-1 alone. The present inventors also showed that combined treatment with FS122m and anti-mPD-1 increased median survival, and increased the percentage of mice with complete tumor regression in the same mouse tumor models compared with the combined increase in median survival, or percentage of mice with complete tumor regression, observed when mice were treated with either FS122m or anti-mPD-1 alone. The present inventors thus showed that the combination of FS122m and anti-mPD-1 enhanced anti-tumor activity, as measured by tumor growth retardation/tumor volume reduction, median survival, and percentage of mice showing complete tumor regression, in E0771, JC and Eph4-1424 mouse tumor models in a synergistic manner.

These nonclinical studies in mouse tumor models support the expectation that the combination of an antibody molecule that binds MSLN and CD137 and a PD-1/PD-L1 inhibitor enhances anti-tumor activity in a synergistic manner. These findings present combination therapy with an antibody molecule that binds MSLN and CD137 and a PD-1/PD-L1 inhibitor as a new therapeutic strategy to improve cancer treatment.

The present inventors were also able to show that the combination of M9657 and the anti-PD-1 antibody pembrolizumab increased T cell activation, tumor cell killing, and cytokine release in vitro in a synergistic manner. This supports the expectation that combination of a bispecific anti-MSLN/CD137 antibody molecule and a PD-1/PD-L1 inhibitor in the treatment of cancer in a human patient will also show a synergistic anti-tumor effect.

Therefore, the present invention provides an antibody molecule that binds MSLN and CD137 for use in a method of treating cancer in a patient, wherein the method comprises administering the antibody molecule that binds MSLN and CD137 in combination with a PD-1/PD-L1 inhibitor. The present invention also relates to a PD-1/PD-L1 inhibitor for use in a method of treating cancer in a patient, wherein the method comprises administering the PD-1/PD-L1 inhibitor in combination with an antibody molecule that binds MSLN and CD137.

The antibody molecule that binds MSLN and CD137 may be an immunoglobulin or an antigen-binding fragment thereof. For example, the antibody molecule may be an IgG, IgA, IgE or IgM molecule, preferably an IgG molecule, such as an IgG1, IgG2, IgG3 or IgG4 molecule, more preferably an IgG1 or IgG2 molecule, most preferably an IgG1 molecule, or a fragment thereof. In a preferred embodiment, the antibody molecule is a complete immunoglobulin molecule.

The antibody molecule may comprise at least one, preferably more than one, complementary determining region (CDR)-based binding site for MSLN and at least one, preferably more than one, binding site for CD137 in a constant domain of the bispecific antibody molecule, preferably in the CH3 domain.

The binding site for CD137 may comprise a first sequence and a second sequence located in the AB and EF structural loops of the CH3 domain of the antibody molecule. Preferably, the first sequence has the sequence set forth in SEQ ID NO: 87. Preferably, the second sequence has the sequence set forth in SEQ ID NO: 88. More preferably, the first sequence has the sequence set forth in SEQ ID NO: 87 and the second sequence has the sequence set forth in SEQ ID NO: 88. According to the IMGT numbering scheme, the first sequence may be located between positions 14 and 17 of the CH3 domain of the antibody molecule. The second sequence may be located between positions 91 and 99 of the CH3 domain of the antibody molecule according to the IMGT numbering scheme. Preferably, the CH3 domain of the antibody molecule has the sequence set forth in SEQ ID NO: 86.

In a preferred embodiment, the bispecific antibody molecule comprises a CH3 domain which comprises, has, or consists of the CH3 domain sequence of FS22-172-003 set forth in SEQ ID NO: 86. The CH3 domain of the bispecific antibody molecule may optionally comprise an additional lysine residue (K) at the immediate C-terminus of the CH3 domain sequence.

A number of Fab regions that bind MSLN are known from WO 2020/011976. The complementary determining region (CDR)-based binding site for MSLN can comprise CDRs 1-6 of any of these Fabs. The antibody molecule that binds MSLN and CD137 may therefore comprise CDRs 1-6 set forth in SEQ 10 ID NOs 4, 6, 8, 12, 14, and 16 [FS28-256-271]; SEQ ID NOs 20, 22, 24, 12, 14 and 28 [FS28-024-052]; SEQ ID NOs 4, 6, 8, 12, 14 and 34 [FS28-256-021]; SEQ ID NOs 4, 6, 8, 12, 14, and 39 [FS28-256-012]; SEQ ID NOs 43, 6, 45, 12, 14 and 34 [FS28-256-023]; SEQ ID NOs 4, 6, 8, 12, 14 and 49 [FS28-256-024]; SEQ ID NOs 43, 6, 45, 12, 14 and 49 [FS28-256-026]; SEQ ID NOs 4, 6, 8, 12, 14 and 16 [FS28-256-027]; SEQ ID NOs 53, 6, 55, 12, 14 and 34 [FS28-256-001]; SEQ ID NOs 53, 6, 55, 12, 14 15 and 49 [FS28-256-005]; SEQ ID NOs 60, 6, 62, 12, 14 and 39 [FS28-256-014]; SEQ ID NOs 43, 6, 45, 12, 14 and 39 [FS28-256-018]; SEQ ID NOs 67, 6, 55, 12, 14 and 39 [FS28-256]; SEQ ID NOs 21, 23, 72, 12, 14 and 28 [FS28-024-051]; SEQ ID NOs 21, 23, 77, 12, 14 and 28 [FS28-024-053]; or SEQ ID NOs 21, 23, 82, 12, 14 and 28 [FS28-024].

A number of bispecific antibody molecules that binds MSLN and CD137 are also known from WO 2020/011976. Antibody M9657 of this application is identical to antibody FS22-172-003-AA/FS28-256-271 of WO 2020/011976. Any of these antibodies can be used and are hereby incorporated by reference. The antibody molecule that binds MSLN and CD137 may therefore comprise the heavy chain and light chain set forth in SEQ ID NOs 2 and 10 (FS22-172-003-AA/FS28-256-271), SEQ ID NOs 18 and 26 25 (FS22-172-003-AA/FS28-024-052), SEQ ID NOs 30 and 32 (FS22-172-003-AA/FS28-256-021), SEQ ID NOs 36 and 37 (FS22-172-003-AA/FS28-256-012), SEQ ID NOs 41 and 32 (FS22-172-003-AA/FS28-256-023), SEQ ID NOs 30 and 47 (FS22-172-003-AA/FS28-256-024), SEQ ID NOs 41 and 47 (FS22-172-003-AA/FS28-256-026), SEQ ID NOs 30 and 10 (FS22-172-003-AA/FS28-256-027), SEQ ID NOS 51 and 32 (FS22-172-003-AA/FS28-256-001), SEQ ID NOs 51 and 47 (FS22-172-003-AA/FS28-256-005), SEQ ID NOs 58 and 37 (FS22-172-003-AA/FS28-256-014), SEQ ID NOs 41 and 37 (FS22-172-003-AA/FS28-256-018), SEQ ID NOs 65 and 37 (FS22-172-003-AA/FS28-256), SEQ ID NOs 70 and 26 (FS22-172-003-AA/FS28-024-051), SEQ ID NOs 75 and 26 (FS22-172-003-AA/FS28-024-053), or SEQ ID NOs 80 and 26 (FS22-172-003-AA/FS28-024), respectively. Preferably, the antibody molecule that binds MSLN and CD137 comprises the heavy chain sequence set forth in SEQ ID NO: 2 and the light chain sequence set forth in SEQ ID NO: 10 (FS22-172-003-AA/FS28-256-271).

PD-1/PD-L1 inhibitors inhibit the PD-1 immune checkpoint. PD-1/PD-L1 inhibitors may inhibit PD-1 activation either directly or indirectly, but preferably inhibit PD-1 activation directly. PD-1 activation may be inhibited directly by inhibition of binding of PD-L1 to PD-1 and/or by inhibition of PD-L1-mediated activation of PD-1. PD-1 activation may be inhibited indirectly through reduction in PD-L1 expression, for example.

PD-1/PD-L1 inhibitors include small molecules, peptides, and antibody molecules. Antibody molecules in this context immunoglobulins, such as IgG1, chimeric antibody molecules, antibody fusion proteins, or antigen-binding antibody fragments, such as scFvs, Fabs, Fcabs, VhHs, monovalent IgGs, diabodies, triabodies, immunoglobulin new antigen receptors (IGNAR), single domain shark variable domains of new antigen receptor (V-NAR), human chimeric IgGs (hcIgGs), minibodies, or nanobodies. In a preferred embodiment, the PD-1/PD-L1 inhibitor is an antibody molecule, or antigen-binding fragment thereof, which binds PD-1 or PD-L1.

The antibody molecule, or antigen-binding fragment thereof, that binds PD-1 may be selected from the group consisting of: nivolumab, pembrolizumab, cemiplimab, dostarlimab, sintilimab, camrelizumab, toripalimab, tislelizumab, spartalizumab, zimberelimab, penpulimab, and candonilimab. In a preferred embodiment, the antibody molecule, or antigen-binding fragment thereof, that binds PD-1 may be selected from the group consisting of: nivolumab, pembrolizumab, and cemiplimab. More preferably the antibody molecule that binds PD-1 is pembrolizumab. The heavy and light chain sequences of pembrolizumab are known in the art and are set forth in SEQ ID NO: 93 and SEQ ID NO: 94, respectively.

The antibody molecule, or antigen-binding fragment thereof, that binds PD-L1 may be selected from the group consisting of: avelumab, atezolizumab, durvalumab, sugemalimab, and envafolimab. Preferably, the antibody molecule, or antigen-binding fragment thereof, that binds PD-L1 is selected from the group consisting of: avelumab, atezolizumab, and durvalumab. More preferably, the antibody molecule that binds PD-L1 is avelumab.

Small molecule inhibitors of PD-1/PD-L1 may be selected from the group consisting of: BMS202, CA-170, fraxinellone, BMS-1166, N-deacetylated BMS-202, BMS-1001 hydrochloride, INCB086550, tomivosertib, PD-1/PD-L1-IN-9, PROTAC PD-1/PD-L1 degrader-1, PD-L1-IN-1, sulfamethoxypyridazine, PD-1-IN-17, PD-1/PD-L1-IN-10, PD-1-IN-24, BMS-8, evixapodlin, PD-1-IN-18, PD-1-IN-17 TFA, BMS-1166 hydrochloride, ARB272572, PD-1, PD-L1-IN-13, sulindac sodium, PD-1/PD-L1-IN 5, PD-1-IN-22, PD-1/PD-L1-IN-NP19, PD-1/PD-L1-IN-27, PD-1-IN-20, PD-1/PD-L1-IN-23, PD-1/PD-L1-IN 5 TFA, PD-1/PD-L1-IN 6, PD-1/PD-L1-IN-14, PD-1/PD-L1-IN-26, PD1-PDL1-IN 1, [D-Leu-4]-OB3, PD-1/PD-L1-IN-16, PD-1/PD-L1-IN-20, PD-1/PD-L1-IN-19, PD-1/PD-L1-IN-17, BMSpep-57, PD-1/PD-L1-IN-24, PD-1/PD-L1-IN-21, HE-S2, PD-1/PD-L1-IN-22, PD-1/PD-L1-IN-18, and PD-1/PD-L1-IN-15.

The membrane-bound protein mesothelin (MSLN) has been shown to be expressed in several cancers. Specifically ovarian cancer, pancreatic adenocarcinoma, mesothelioma, and non-small cell lung carcinomas have been shown to express high levels of MSLN. The present inventors found that this is also the case for cervical carcinoma. Without wishing to be bound by theory, it is thought that binding of the antibody molecule to MSLN results in antibody crosslinking, binding to CD137 expressed at the surface of an immune cell, followed by CD137 clustering and activation, ultimately resulting in activation of the immune cell.

Accordingly, the cancer to be treated is preferably a cancer that expresses, or has been determined to express, MSLN. Preferably, the cancer is selected from the group consisting of ovarian cancer, pancreatic adenocarcinoma, mesothelioma, cervical carcinoma, and non-small cell lung cancer.

Combination of FS122m and anti-mPD-1 resulted in greater anti-tumor activity in E0771, JC and Eph4-1424 mouse tumor models than the combined anti-tumor activity observed when mice were treated with either FS122m or anti-PD-1 alone, as explained above. In other words, the anti-tumor effect of the combination was synergistic.

Therefore, in one embodiment, treatment with the antibody molecule that binds MSLN and CD137 in combination with the PD-1/PD-L1 inhibitor results in a greater anti-tumor effect than the anti-tumor effect than the anti-tumor effect observed when patients are treated with either the antibody molecule that binds MSLN and CD137, or the PD-1/PD-L1 inhibitor, alone. Preferably, treatment with the antibody molecule that binds MSLN and CD137 in combination with the PD-1/PD-L1 inhibitor results in a greater anti-tumor effect than the combined anti-tumor effect observed when patients are treated with either the antibody molecule that binds MSLN and CD137 or the PD-1/PD-L1 inhibitor alone. The anti-tumor effect may be tumor growth inhibition or retardation. The anti-tumor effect may thus be a reduction in the tumor volume. The anti-tumor effect may be an increase in median survival of the patient. The anti-tumor effect may be an increase in the percentage of patients experiencing complete tumor regression, such as a clinical complete response, or a pathological complete response. The determination of these anti-tumor effects is within the capabilities of the skilled person.

The bispecific antibody molecule that binds MSLN and CD137 and the PD-1/PD-L1 inhibitor can be administered to a subject by any suitable means. Accordingly, in one embodiment the antibody molecule that binds MSLN and CD137 and/or the PD-1/PD-L1 inhibitor is administered parenterally. The antibody molecule that binds MSLN and CD137 and/or the PD-1/PD-L1 inhibitor may be administered intravenously, intramuscularly, subcutaneously, intraperitoneally or spinally. Alternatively, the antibody molecule that binds MSLN and CD137 and/or the PD-1/PD-L1 inhibitor may be administered by injection or infusion.

The antibody molecule that binds MSLN and CD137 and/or the PD-1/PD-L1 inhibitor may be administered non-parenterally. The antibody molecule that binds MSLN and CD137 and/or the PD-1/PD-L1 inhibitor may be administered orally, intranasally, vaginally, rectally, sublingually, or topically.

The antibody molecule that binds MSLN and CD137 and the PD-1/PD-L1 inhibitor can be part of the same formulation or part of separate formulations, but preferably are provided as separate formulations. Accordingly, the antibody molecule that binds MSLN and CD137 and the PD-1/PD-L1 inhibitor may be administered to the patient concomitantly or sequentially, but preferably administered sequentially.

Where the antibody molecule that binds MSLN and CD137 and the PD-1/PD-L1 inhibitor are administered to the patient sequentially, they are preferably administered to the patient within 4 days of each other, more preferably within 3 days of each other, more preferably within 2 days of each other, or sequentially on the same day.

The present invention also provides a method of treating cancer comprising administering to the individual in need an antibody molecule that binds MSLN and CD137 and a PD-1/PD-L1 inhibitor. Preferably, the method of treating cancer comprises administering to the individual in need thereof a therapeutically effective amount of the antibody molecule that binds MSLN and CD137 and a therapeutically effective amount of the PD-1/PD-L1 inhibitor. In one embodiment, the method may comprise determining whether a cancer in a patient expresses MSLN and treating the patient if the cancer has been determined to express MSLN. Alternatively, the method may comprise a step of ordering the results of a test determining whether a cancer in a patient expresses MSLN and treating the patient if the test results show that the cancer expresses MSLN.

The present invention also provides a use of an antibody molecule that binds MSLN and CD137 for the manufacture of a medicament for the treatment of cancer, wherein the antibody molecule that binds MSLN and CD137 is administered in combination with a PD-1/PD-L1 inhibitor. The present invention also provides a use of a PD-1/PD-L1 inhibitor for the manufacture of a medicament for the treatment of cancer, wherein the PD-1/PD-L1 inhibitor is administered in combination with an antibody molecule that binds MSLN and CD137.

The present invention also provides a kit comprising an antibody molecule that binds MSLN and CD137 and a pharmaceutically acceptable excipient and a PD-1/PD-L1 inhibitor and a pharmaceutically acceptable excipient.

Thus the present invention provides:
[1] An antibody molecule that binds MSLN and CD137 for use in a method of treating cancer in a patient, wherein the method comprises administering the antibody in combination with a PD-1/PD-L1 inhibitor.
[2] A PD-1/PD-L1 inhibitor for use in a method of treating cancer in a patient, wherein the method comprises administering the PD-1/PD-L1 inhibitor in combination with an antibody molecule that binds MSLN and CD137.
[3] A method of treating cancer in an individual, the method comprising administering to the individual an antibody molecule that binds MSLN and CD137 and a PD-1/PD-L1 inhibitor.
[4] Use of an antibody molecule that binds MSLN and CD137 for the manufacture of a medicament for the treatment of cancer, wherein the antibody molecule that binds MSLN and CD137 is administered in combination with a PD-1/PD-L1 inhibitor.
[5] Use of a PD-1/PD-L1 inhibitor for the manufacture of a medicament for the treatment of cancer, wherein the PD-1/PD-L1 inhibitor is administered in combination with an antibody molecule that binds MSLN and CD137
[6] A kit comprising
   (a) an antibody molecule that binds MSLN and CD137 and a pharmaceutically acceptable excipient; and
   (b) a PD-1/PD-L1 inhibitor and a pharmaceutically acceptable excipient.
[7] The antibody molecule or PD-1/PD-L1 inhibitor for use, method, use, or kit according to [1] to [6], wherein the antibody molecule that binds MSLN and CD137 comprises:
   (a) a complementary determining region (CDR)-based antigen-binding site for MSLN; and
   (b) a CD137 antigen-binding site located in a CH3 domain of the antibody molecule.
[8] The antibody molecule or PD-1/PD-L1 inhibitor for use, method, use, or kit according to [7], wherein the antibody molecule that binds MSLN and CD137 comprises two or more of
   (a) a complementary determining region (CDR)-based antigen-binding site for MSLN; and
   (b) a CD137 antigen-binding site located in a CH3 domain of the antibody molecule.
[9] The antibody molecule or PD-1/PD-L1 inhibitor for use, method, use, or kit according to [1] to [8], wherein the antibody molecule that binds MSLN and CD137 is an IgG, IgA, IgE, IgM molecule, or an antigen-binding fragment thereof.
[10] The antibody molecule or PD-1/PD-L1 inhibitor for use, method, use, or kit according to [9], wherein the antibody molecule that binds MSLN and CD137 is an IgG molecule, or an antigen-binding fragment thereof.
[11] The antibody molecule or PD-1/PD-L1 inhibitor for use, method, use, or kit according to [10], wherein the antibody molecule that binds MSLN and CD137 is an IgG1 or IgG2 molecule, or an antigen-binding fragment thereof.
[12] The antibody molecule or PD-1/PD-L1 inhibitor for use, method, use, or kit according to [11], wherein the antibody molecule that binds MSLN and CD137 is an IgG1 molecule, or an antigen-binding fragment thereof.
[13] The antibody molecule or PD-1/PD-L1 inhibitor for use, method, use, or kit according to [7] to [12], wherein the CDR-based antigen-binding site for MSLN comprises CDRs 1-6 set forth in:
   (i) SEQ ID NOs 4, 6, 8, 12, 14, and 16, respectively [FS28-256-271];
   (ii) SEQ ID NOs 20, 22, 24, 12, 14 and 28, respectively [FS28-024-052];
   (iii) SEQ ID NOs 4, 6, 8, 12, 14 and 34, respectively [FS28-256-021];
   (iv) SEQ ID NOs 4, 6, 8, 12, 14, and 39, respectively [FS28-256-012];
   (v) SEQ ID NOs 43, 6, 45, 12, 14 and 34, respectively [FS28-256-023];
   (vi) SEQ ID NOs 4, 6, 8, 12, 14 and 49, respectively [FS28-256-024];
   (vii) SEQ ID NOs 43, 6, 45, 12, 14 and 49, respectively [FS28-256-026];
   (viii) SEQ ID NOs 4, 6, 8, 12, 14 and 16, respectively [FS28-256-027];
   (ix) SEQ ID NOs 53, 6, 55, 12, 14 and 34, respectively [FS28-256-001];
   (x) SEQ ID NOs 53, 6, 55, 12, 14 and 49, respectively [FS28-256-005];
   (xi) SEQ ID NOs 60, 6, 62, 12, 14 and 39, respectively [FS28-256-014];
   (xii) SEQ ID NOs 43, 6, 45, 12, 14 and 39, respectively [FS28-256-018];
   (xiii) SEQ ID NOs 67, 6, 55, 12, 14 and 39, respectively [FS28-256];
   (xiv) SEQ ID NOs 21, 23, 72, 12, 14 and 28, respectively [FS28-024-051];
   (XV) SEQ ID NOs 21, 23, 77, 12, 14 and 28, respectively [FS28-024-053]; or
   (xvi) SEQ ID NOs 21, 23, 82, 12, 14 and 28, respectively [FS28-024]; and
      wherein the CD137 antigen-binding site comprises a first sequence and a second sequence located in the AB and EF structural loops of the CH3 domain, respectively, wherein the first and second sequence have the sequence set forth in SEQ ID NOs 87 and 88, respectively.
[14] The antibody molecule or PD-1/PD-L1 inhibitor for use, method, use, or kit according to [13],
   wherein
   (i) the first sequence is located between positions 14 and 17 of the CH3 domain of the antibody molecule; and/or
   (ii) wherein the second sequence is located between 91 and 99 of the CH3 domain of the antibody molecule; and
   wherein the amino acid residue numbering is according to the IMGT numbering scheme.
[15] The antibody molecule or PD-1/PD-L1 inhibitor for use, method, use, or kit according to [1] to [14], wherein the antibody molecule that binds MSLN and CD137 comprises the CH3 domain sequence set forth in SEQ ID NO: 86.
[16] The antibody molecule or PD-1/PD-L1 inhibitor for use, method, use, or kit according to [15], wherein the CH3 domain comprises an additional lysine residue (K) at the immediate C-terminus of the CH3 domain sequence.
[17] The antibody molecule or PD-1/PD-L1 inhibitor for use, method, use, or kit according to [1] to [16], wherein the antibody molecule that binds MSLN and CD137 comprises the heavy chain and light chain of antibody:
   (i) FS22-172-003-AA/FS28-256-271 set forth in SEQ ID NOs 2 and 10, respectively;
   (ii) FS22-172-003-AA/FS28-024-052 set forth in SEQ ID NOs 18 and 26, respectively;
   (iii) FS22-172-003-AA/FS28-256-021 set forth in SEQ ID NOs 30 and 32, respectively;
   (iv) FS22-172-003-AA/FS28-256-012 set forth in SEQ ID NOs 36 and 37, respectively;
   (v) FS22-172-003-AA/FS28-256-023 set forth in SEQ ID NOs 41 and 32, respectively;
   (vi) FS22-172-003-AA/FS28-256-024 set forth in SEQ ID NOs 30 and 47, respectively;
   (vii) FS22-172-003-AA/FS28-256-026 set forth in SEQ ID NOs 41 and 47, respectively;
   (viii) FS22-172-003-AA/FS28-256-027 set forth in SEQ ID NOs 30 and 10, respectively;
   (ix) FS22-172-003-AA/FS28-256-001 set forth in SEQ ID NOs 51 and 32, respectively;
   (x) FS22-172-003-AA/FS28-256-005 set forth in SEQ ID NOs 51 and 47, respectively;
   (xi) FS22-172-003-AA/FS28-256-014 set forth in SEQ ID NOs 58 and 37, respectively;
   (xii) FS22-172-003-AA/FS28-256-018 set forth in SEQ ID NOs 41 and 37, respectively;
   (xiii) FS22-172-003-AA/FS28-256 set forth in SEQ ID NOs 65 and 37, respectively;
   (xiv) FS22-172-003-AA/FS28-024-051 set forth in SEQ ID NOs 70 and 26, respectively;
   (xv) FS22-172-003-AA/FS28-024-053 set forth in SEQ ID NOs 75 and 26, respectively;
   or
   (xvi) FS22-172-003-AA/FS28-024 set forth in SEQ ID NOs 80 and 26, respectively
[18] The antibody molecule or PD-1/PD-L1 inhibitor for use, method, use, or kit according to [1] to [17], wherein the antibody molecule that binds MSLN and CD137 comprises the heavy chain sequence set forth in SEQ ID NO: 2 and the light chain sequence set forth in SEQ ID NO: 10 [FS22-172-003-AA/FS28-256-271].
[19] The antibody molecule or PD-1/PD-L1 inhibitor for use, method, use, or kit according to [1] to [18], wherein the PD-1/PD-L1 inhibitor is selected from the group consisting of a small molecule inhibitor, a peptide, an antibody, a chimeric antibody, an antibody fusion protein, and antigen-binding fragments thereof.

[20] The antibody molecule or PD-1/PD-L1 inhibitor for use, method, use, or kit according to [19], wherein the antigen-binding fragment is selected from the group consisting of a scFv, Fab, Fcab, VhH, monovalent IgG, di- or triabody, immunoglobulin new antigen receptor (IGNAR), single domain shark variable domain of new antigen receptor (V-NAR), hIgG, minibody or a nanobody.
[21] The antibody molecule or PD-1/PD-L1 inhibitor for use, method, use, or kit according to [1] to [20], wherein the PD-1/PD-L1 inhibitor is an antibody, or antigen-binding fragment thereof, that binds PD-1 or PD-L1.
[22] The antibody molecule or PD-1/PD-L1 inhibitor for use, method, use, or kit according to [21], wherein the antibody, or antigen-binding fragment thereof, that binds PD-1 or PD-L1 is an IgG, IgA, IgE, IgM molecule, or fragment thereof.
[23] The antibody molecule or PD-1/PD-L1 inhibitor for use, method, use, or kit according to or [22], wherein the antibody, or antigen-binding fragment thereof, that binds PD-1 is selected from the group consisting of nivolumab, pembrolizumab and cemiplimab.
[24] The antibody molecule or PD-1/PD-L1 inhibitor for use, method, use, or kit according to [23], wherein the antibody, or antigen-binding fragment thereof, that binds PD-1 is pembrolizumab.
[25] The antibody molecule or PD-1/PD-L1 inhibitor for use, method, use, or kit according to or [22], wherein the antibody, or antigen-binding fragment thereof, that binds PD-L1 is selected from the group consisting of avelumab, atezolizumab and durvalumab.
[26] The antibody molecule or PD-1/PD-L1 inhibitor for use, method, use, or kit according to [25], wherein the antibody, or antigen-binding fragment thereof, that binds PD-L1 is avelumab.
[27] The antibody molecule or PD-1/PD-L1 inhibitor for use, method, use, or kit according to [1] to [26], wherein the cancer expresses MSLN, or has been determined to express MSLN.
[28] The antibody molecule or PD-1/PD-L1 inhibitor for use, method, use, or kit according to [1] to [27], wherein the cancer is selected from the group consisting of ovarian cancer, pancreatic adenocarcinoma, mesothelioma, cervical carcinoma and non-small cell lung carcinoma.
[29] The antibody molecule or PD-1/PD-L1 inhibitor for use, method, use, or kit according to [1] to [28], wherein treatment with the antibody molecule that binds MSLN and CD137 and the PD-1/PD-L1 inhibitor results in greater anti-tumor activity than monotherapy treatment with the antibody molecule that binds MSLN and CD137 or monotherapy treatment with the PD-1/PD-L1 inhibitor.
[30] The antibody molecule or PD-1/PD-L1 inhibitor for use, method, use, or kit according to [1] to [29], wherein treatment with the antibody molecule that binds MSLN and CD137 and the PD-1/PD-L1 inhibitor results in greater anti-tumor activity than the combined anti-tumor activity of monotherapy treatment with the antibody molecule that binds MSLN and CD137 and monotherapy treatment with the PD-1/PD-L1 inhibitor.
[31] The antibody molecule or PD-1/PD-L1 inhibitor for use, method, use, or kit according to [1] to [30], wherein treatment with the antibody molecule that binds MSLN and CD137 and the PD-1/PD-L1 inhibitor results in greater tumor growth retardation, tumor volume reduction, median survival and/or the number of complete tumor regressions than monotherapy treatment with the antibody molecule that binds MSLN and CD137 or monotherapy treatment with the PD-1/PD-L1 inhibitor.
[32] The antibody molecule or PD-1/PD-L1 inhibitor for use, method, use, or kit according to [1] to [31], wherein treatment with the antibody molecule that binds MSLN and CD137 and the PD-1/PD-L1 inhibitor results in greater tumor growth retardation, tumor volume reduction, median survival and/or the number of complete tumor regressions than the combined tumor growth retardation, tumor volume reduction, median survival increase and/or increase in the number of complete tumor regressions of monotherapy treatment with the antibody molecule that binds MSLN and CD137 and monotherapy treatment with the PD-1/PD-L1 inhibitor.
[33] The antibody molecule or PD-1/PD-L1 inhibitor for use, method, use, or kit according to [1] to [32], wherein the antibody molecule that binds MSLN and CD137 and/or the PD-1/PD-L1 inhibitor is administered parenterally.
[34] The antibody molecule or PD-1/PD-L1 inhibitor for use, method, use, or kit according to [33], wherein the antibody molecule that binds MSLN and CD137 and/or the PD-1/PD-L1 inhibitor is administered intravenously, intramuscularly, subcutaneously, intraperitoneally or spinally
[35] The antibody molecule or PD-1/PD-L1 inhibitor for use, method, use, or kit according to or [34], wherein the antibody molecule that binds MSLN and CD137 and/or the PD-1/PD-L1 inhibitor is administered by injection or infusion.
[36] The antibody molecule or PD-1/PD-L1 inhibitor for use, method, use, or kit according to [1] to [32], wherein the antibody molecule that binds MSLN and CD137 and/or the PD-1/PD-L1 inhibitor is administered non-parenterally.
[37] The antibody molecule or PD-1/PD-L1 inhibitor for use, method, use, or kit according to [36], wherein the antibody molecule that binds MSLN and CD137 and/or the PD-1/PD-L1 inhibitor is administered orally, intranasally, vaginally, rectally, sublingually, or topically.
[38] The antibody molecule or PD-1/PD-L1 inhibitor for use, method, use, or kit according to [1] to [37], wherein the antibody molecule that binds MSLN and CD137 and the PD-1/PD-L1 inhibitor are administered to the patient concomitantly or sequentially.
[39] The antibody molecule or PD-1/PD-L1 inhibitor for use, method, use, or kit according to [38], wherein the antibody molecule that binds MSLN and CD137 and the PD-1/PD-L1 inhibitor are administered to the patient sequentially.
[40] The antibody molecule or PD-1/PD-L1 inhibitor for use, method, use, or kit according to [39], wherein the antibody molecule that binds MSLN and CD137 and the PD-1/PD-L1 inhibitor are to the patient within no more than 4 days of each other.
[41] The antibody molecule or PD-1/PD-L1 inhibitor for use, method, use, or kit according to or [40], wherein the antibody molecule that binds MSLN and CD137 and the PD-1/PD-L1 inhibitor are to the patient within no more than 3 days of each other.
[42] The antibody molecule or PD-1/PD-L1 inhibitor for use, method, use, or kit according to [39] to [41], wherein the antibody molecule that binds MSLN and CD137 and the PD-1/PD-L1 inhibitor are to the patient within no more than 2 days of each other.
[43] The antibody molecule or PD-1/PD-L1 inhibitor for use, method, use, or kit according to [39] to [42], wherein the antibody molecule that binds MSLN and CD137 and the PD-1/PD-L1 inhibitor are to the patient on the same day.
[44] The antibody molecule or PD-1/PD-L1 inhibitor for use, or the method according to any one of [1] to [3] and [7] to [43], wherein the method comprises determining whether the cancer expresses MSLN and treating the individual if the cancer expresses MSLN.

### SUMMARY OF THE FIGURES

Embodiments and experiments illustrating the principles of the invention will now be discussed with reference to the accompanying figures in which:

FIG. 1 shows the results of a bioluminescence report assay in which the bioluminescence signal was measured as a function of NF-κB expression in PD-1⁺CD137 effector cells co-cultured with PD-L1 aAPC/CHO-K1- and MSLN-expressing CHO tumor cells. Cells were treated with different concentrations of either M9657, M9657+pembrolizumab, M9657+anti-HEL-hIgG1-LALA antibody (isotype control), CD137L+pembrolizumab, or CD137L+anti-HEL-hIgG1-LALA antibody. Treatment with M9657+pembrolizumab resulted in a significantly higher bioluminescence signal than either of M9657, M9657+anti-HEL-hIgG1-LALA antibody, CD137L+pembrolizumab, or CD137L+anti-HEL-hIgG1-LALA antibody. Mean±standard error of the mean (SEM) is shown, and data represent 3 replicate experiments.

FIG. 2 shows the results of an assay measuring cytotoxicity of target cells and cytokine release of CD8⁺ T cells co-cultured with NCI-H226 tumor target cells. Cytotoxicity was measured as % killing of the target cells (A) and the normalized area under the curve (AUC) of the results in (A) (B). Cytokine release was measured as INFγ levels in the supernatant (C) and the normalized area under the curve (AUC) of the results in (C) (D). Cells were treated with different concentrations of either M9657+pembrolizumab, M9657, anti-HEL-hIgG1-LALA antibody (isotype control), or BiTE (anti-CD3×anti-EGFR BiTE). Treatment with M9657+pembrolizumab increased the cytotoxicity of CD8+ T cells compared with M9657, anti-HEL-hIgG1-LALA antibody, or BiTE (anti-CD3×anti-EGFR BiTE) monotherapy. % Killing (A) and AUC (B) was measured in cells from five different donors and presented as mean±SEM. IFNγ concentration (C) and AUC (D) was measured in cells from six different donors and presented as mean±SEM.

FIG. 3 shows efficacy of treatment in a E0771 orthotopic mouse breast cancer model in C57BL/6 mice. Progression in the mouse cancer model was measured as average tumor volume over time (A), median survival (B), % body weight change (C) and changes in individual tumor volume over time (D). Mice were treated with either anti-HEL-hIgG1-LALA antibody (isotype control), FS122m, anti-mPD-1, or FS122m+anti-mPD-1. Treatment with FS122m+anti-mPD-1 showed a reduction in average tumor volume over time, while FS122m and anti-mPD-1 monotherapy merely retarded tumor growth compared to the anti-HEL-hIgG1-LALA isotype control over the course of the study (A). FS122m and anti-mPD-1 combined treatment also enhanced median survival relative to FS122m and anti-mPD-1 monotherapy (B) and induced complete tumor regression in 7 of 9 mice compared with no complete tumor regression in 9 mice treated with FS122m monotherapy and complete tumor regression in only 1 of 9 mice treated with anti-mPD-1 monotherapy. Changes in body weight were comparable between all treatments including the anti-HEL-hIgG1-LALA isotype control, demonstrating that all treatments were well tolerated (C). Tumor volume data were log-transformed and two-way analysis of variance (ANOVA) was performed followed by Tukey's multiple comparison test, where **=P≤0.01, ***=P≤0.001, ****=P≤0.0001. Survival is presented as median percentage survival and average tumor volume and body weight change are shown as mean±SEM.

FIG. 4 shows efficacy of treatment in a JC subcutaneous mouse breast tumor model in BALB/c mice. Progression in the mouse cancer model was measured as average tumor volume over time (A), median survival (B), % body weight change (C) and changes in individual tumor volume over time (D). Mice were treated with either anti-HEL-hIgG1-LALA, FS122m, anti-mPD-1, or FS122m+anti-mPD-1. Over the course of the study, treatment with FS122m+anti-mPD-1 retarded tumor growth to a much larger extent than FS122m and anti-mPD-1 monotherapy when compared with the anti-HEL-hIgG1-LALA isotype control (A). FS122m and anti-mPD-1 combined treatment also enhanced median survival relative to FS122m and anti-mPD-1 monotherapy (B) and induced complete tumor regression in 3 of 10 mice compared with no mice achieving complete tumor regression when treated with FS122m monotherapy or anti-mPD-1 monotherapy. Changes in body weight were comparable between all treatments including the anti-HEL-hIgG1-LALA isotype control, demonstrating that all treatments were well tolerated (C). Tumor volume data were log-transformed and two-way analysis of variance (ANOVA) was performed followed by Tukey's multiple comparison test, where **=P≤0.01, ***=P≤0.001, ****=P≤0.0001. Survival is presented as median percentage survival and average tumor volume and body weight change are shown as mean±SEM.

FIG. 5 shows efficacy of treatment in a Eph4-1424 subcutaneous mouse breast tumor model in BALB/c mice. Progression in the mouse cancer mouse model was measured as average tumor volume over time (A), median survival (B), % body weight change (C) and changes in individual tumor volume over time (D). Mice were treated with either anti-HEL-hIgG1-LALA, FS122m, anti-mPD-1, or FS122m+anti-mPD-1. Treatment with FS122m+anti-mPD-1 showed a reduction in average tumor volume over time, while FS122m and anti-mPD-1 monotherapy merely retarded tumor growth compared with treatment with the anti-HEL-hIgG1-LALA isotype control over the course of the study (A). Combined treatment with FS122m and anti-mPD-1 also enhanced median survival relative to FS122m and anti-mPD-1 monotherapy (B) and induced complete tumor regression in 10 of 10 mice, while complete tumor regression was observed in only 1 of 10 mice treated with FS122m monotherapy and 6 of 10 mice treated with anti-mPD-1 monotherapy. Changes in body weight were comparable between all treatments, including the anti-HEL-hIgG1-LALA isotype control, demonstrating that all treatments were well tolerated (C). Tumor volume data were log-transformed and two-way analysis of variance (ANOVA) was performed followed by Tukey's multiple comparison test, where **=P≤0.01, ***=P≤0.001, ****=P≤0.0001. Survival is presented as median percentage survival and average tumor volume and body weight change are shown as mean±SEM.

### DETAILED DESCRIPTION OF THE INVENTION

The present invention relates to an antibody molecule that binds MSLN and CD137 for use in the treatment of cancer in combination with a PD-1/PD-L1 inhibitor. The present invention also relates to a PD-1/PD-L1 inhibitor for use in the treatment of cancer in combination with an antibody molecule that binds MSLN and CD137.

The term “PD-1/PD-L1 inhibitor” describes a molecule that inhibits the PD-1 immune checkpoint. PD-1/PD-L1 inhibitors may inhibit PD-1 activation either directly or indirectly, but preferably inhibit PD-1 activation directly. PD-1 activation may be inhibited directly by inhibition of binding of PD-L1 to PD-1 and/or by inhibition of PD-L1-mediated activation of PD-1. PD-1 activation may be inhibited indirectly through reduction in PD-L1 expression, for example. The PD-1/PD-L1 inhibitor may be a small molecule inhibitor, peptide, antibody, chimeric antibody, antibody fusion protein, or antibody fragment such as a scFv, Fab, Fcab, VhH, monovalent IgG, di- or triabody, IGNAR, V-NAR, hcIgG, minibody, or nanobody. For example, the PD-1/PD-L1 inhibitor may be an antibody, or antigen-binding fragment thereof, which binds to PD-1 or PD-L1.

The terms “antibody molecule” describe an immunoglobulin whether natural or partly or wholly synthetically produced. The antibody molecule may be human or humanised, preferably human. The antibody molecule may preferably be a monoclonal antibody. Examples of antibody molecules are the immunoglobulin isotypes, such as immunoglobulin G, M, A, E and D, and their isotypic subclasses, such as IgG1, IgG2, IgG3 and IgG4, as well as antigen-binding fragments thereof. The antibody molecules may be isolated, in the sense of being free from contaminants, such as antibody molecules able to bind other polypeptides and/or serum components.

The PD-1/PD-L1 inhibitor may be natural or partly or wholly synthetically produced. For example, the PD-1/PD-L1 inhibitor may be a recombinant antibody molecule.

The antibody or antigen-binding fragment thereof that binds to PD-1 or PD-L1 may be an anti-PD-1 antibody or antigen-binding fragment thereof. Preferably, the anti-PD-1 antibody is selected from the list consisting of nivolumab, pembrolizumab and cemiplimab. More preferably, the anti-PD-1 antibody is pembrolizumab.

The antibody or antigen-binding fragment thereof that binds to PD-1 or PD-L1 may be an anti-PD-L1 antibody or antigen-binding fragment thereof. Preferably, the anti-PD-1 antibody is selected from the list consisting of avelumab, atezolizumab and durvalumab. More preferably, the anti-PD-L1 antibody is avelumab.

Antibodies and methods for their construction and use are well-known in the art and are described in, for example, Holliger and Hudson, 2005. It is possible to take monoclonal and other antibodies and use techniques of recombinant DNA technology to produce other antibodies or chimeric molecules which retain the specificity of the original antibody. Such techniques may involve introducing CDRs or variable regions of one antibody molecule into a different antibody molecule (EP-A-184187, GB 2188638A and EP-A-239400). New antibodies against known targets can be routinely produced and can arrived at without undue burden by the person skilled in the art.

Accordingly, the PD-1/PD-L1 inhibitor may be any anti-PD-1/PD-L1 antibody not listed above which is comprised in the state of the art or which is not yet comprised in the state of the art and can be arrived at using commonly available techniques known in the art.

In the following, the term “bispecific antibody molecule” is used to refer to the antibody molecule which binds MSLN and CD137.

The bispecific antibody molecule may be an immunoglobulin or an antigen-binding fragment thereof. In one embodiment, the bispecific antibody molecule binds to MSLN and CD137 independently. In one embodiment, the bispecific antibody binds MSLN and CD137 concomitantly.

The term “bispecific” refers to a molecule that will not show any significant binding to molecules other than its two specific binding partners. The term may also refer to specific epitopes of the two binding partners, which may be carried by other antigens, in which case the antibody may also bind to the antigens carrying the specific epitopes. In a preferred embodiment, the bispecific antibody molecule does not show any significant binding activity to OX40, GITR, CD40, CEACAM-5, E-Cadherin, Thrombomodulin, or EpCAM.

The bispecific antibody molecule may be natural or partly or wholly synthetically produced. For example, the antibody molecule may be a recombinant antibody molecule.

The bispecific antibody molecule may comprise at least one, preferably more than one, complementary determining region (CDR)-based binding site for MSLN and at least one, preferably more than one, binding site for CD137 in a constant domain of the bispecific antibody molecule, preferably at least one CH3 domain.

The bispecific antibody molecule may be an immunoglobulin or an antigen-binding fragment thereof. For example, the bispecific antibody molecule may be an IgG, IgA, IgE or IgM molecule, preferably an IgG molecule, such as an IgG1, IgG2, IgG3 or IgG4 molecule, more preferably an IgG1 or IgG2 molecule, most preferably an IgG1 molecule, or an antigen-binding fragment thereof. In a preferred embodiment, the bispecific antibody molecule is a complete immunoglobulin molecule.

In other embodiments, the bispecific antibody molecule may be an antigen-binding fragment comprising a CDR-based antigen-binding site for MSLN and an antigen-binding site for CD137 located in a constant domain. For example, the antigen-binding fragment may be a scFv-Fc fusion where the scFv binds to MSLN and the Fc binds to CD137 or a minibody, which comprises an scFv joined to a CH3 domain (Hu et al. (1996), Cancer Res., 56 (13): 3055-61).

In a preferred embodiment, the bispecific antibody molecule is a mAb² (TM) bispecific antibody. A mAb² bispecific antibody, as referred to herein, is an IgG immunoglobulin which includes a CDR-based antigen binding site in each of its variable regions and at least one antigen binding site in a constant domain of the antibody molecule.

A number of antibody molecules that bind MSLN and CD137 are known from WO 2020/011976. Antibody M9657 of this application is identical to antibody FS22-172-003-AA/FS28-256-271 of WO 2020/011976. Any of these antibodies can be used. The CDR-based antigen-binding site of the bispecific antibody molecule may therefore comprise the three VH CDRs or three VL CDRs, preferably the three VH CDRs and the three VL CDRs, of antibody FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014,FS22-172-003-AA/FS28-256-018, FS22-172-003-AA/FS28-256, FS22-172-003-AA/FS28-024-051, FS22-172-003-AA/FS28-024-053, or FS22-172-003-AA/FS28-024, preferably antibody FS22-172-003-AA/FS28-256-271 or FS22-172-003-AA/FS28-024-052, most preferably antibody FS22-172-003-AA/FS28-256-271.

The sequences of the CDRs may be readily determined from the VH and VL domain sequences of an antibody molecule using routine techniques. The VH and VL domain sequences of antibodies FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026,FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, and FS22-172-003-AA/FS28-024 are described herein, and the three VH and three VL domain CDRs of said antibodies may thus be determined from said sequences. The CDR sequences may, for example, be determined according to Kabat et al., 1991 or the international ImMunoGeneTics information system (IMGT) (Lefranc et al., 2015).

The bispecific antibody molecule may carry a LALA mutation or not. In a preferred embodiment, the bispecific antibody molecule carries a LALA mutation. LALA mutation describes a type of mutation for disrupting the antibody effector function of an antibody molecule or fragment thereof. The LALA mutation is associated with several favourable antibody properties such as reduced toxicity (Lo et al. (2017), The Journal of Biological Chemistry, 292 (9): 3900-3908). The mutation eliminates binding of the antibody molecule or fragment thereof to Fcγ-receptors and is located in the CH2 domain. The sequences of the VH domain and VL domain, and therefore of the VH domain CDR1, CDR2 and CDR3 and the VL domain CDR1, CDR2 and CDR3, of an antibody containing the LALA mutation are the same as an antibody which does not contain the LALA mutation. The LALA mutation involves substitution of the leucine residues at positions 1.3 and 1.2 of the CH2 domain according to the IMGT numbering scheme with alanine (L1.3A and L1.2A). According to the Kabat numbering system, the LALA mutation constitutes a L247A L248A substitution. Alternatively, complement activation (C1q binding) and ADCC are known to be reduced through mutation of the proline at position 114 of the CH2 domain to alanine or glycine according to the IMGT numbering system (P114A or P114G) (Idusogie et al., 2000; Klein et al., 2016). According to the Kabat numbering system, this mutation constitutes a P348A or P348G substitution. These two types of mutations may also be combined in order to generate antibody molecules with further reduced or no ADCC or CDC activity.

Accordingly, the bispecific antibody molecule may comprise a CH2 domain, wherein the CH2 domain comprises an alanine residue at position 1.3 and an alanine residue at position 1.2, wherein the amino acid numbering is according to the IMGT numbering system. The bispecific antibody molecule may comprise a CH2 domain, wherein the CH2 domain comprises an alanine residue at position 247 and an alanine residue at position 248, wherein the amino acid numbering is according to the Kabat numbering system. For example, the CH2 domain may have the amino acid sequence set forth in SEQ ID NO: 90. In another embodiment, the antibody molecule may comprise a CH2, wherein the CH2 domain comprises an alanine residue at position 114. For example, the CH2 domain may have the amino acid sequence set forth in SEQ ID NO: 91. In another embodiment, the antibody molecule may comprise a CH2, wherein the CH2 domain comprises an alanine residue at position 1.3, an alanine residue at position 1.2 and an alanine residue at position 114. For example, the CH2 domain may have the amino acid sequence set forth in SEQ ID NO: 92.

The VH domain CDR1, CDR2 and CDR3 sequences of the bispecific antibody molecule according to IMGT numbering may be the sequences located at positions 27-38, 56-65, and 105-117, of the VH domain of the antibody molecule, respectively.

The VH domain CDR1, CDR2 and CDR3 sequences of the bispecific antibody molecule according to Kabat numbering may be the sequences at located positions 31-35, 50-65, and 95-102 of the VH domain, respectively.

The VL domain CDR1, CDR2 and CDR3 sequences of the bispecific antibody molecule according to IMGT numbering may be the sequences located at positions 27-38, 56-65, and 105-117, of the VL domain, respectively.

The VL domain CDR1, CDR2 and CDR3 sequences of the bispecific antibody molecule according to Kabat numbering may be the sequences at located positions 24-34, 50-56, and 89-97 of the VL domain, respectively.

For example, the sequence of the VH domain CDR1, CDR2 and CDR3 of:
(i) FS22-172-003-AA/FS28-256-271 may be as set forth in SEQ ID NOs 4, 6, and 8, respectively;
(ii) FS22-172-003-AA/FS28-024-052 may be as set forth in SEQ ID NOs 20, 22, and 24, respectively;
(iii) FS22-172-003-AA/FS28-256-021 may be as set forth in SEQ ID NOs 4, 6, and 8, respectively;
(iv) FS22-172-003-AA/FS28-256-012 may be as set forth in SEQ ID NOs 4, 6, and 8, respectively;
(v) FS22-172-003-AA/FS28-256-023 may be as set forth in SEQ ID NOs 42, 6, and 44, respectively;
(vi) FS22-172-003-AA/FS28-256-024 may be as set forth in SEQ ID NOs 4, 6, and 8, respectively;
(vii) FS22-172-003-AA/FS28-256-026 may be as set forth in SEQ ID NOs 43, 6, and 45, respectively;
(viii) FS22-172-003-AA/FS28-256-027 may be as set forth in SEQ ID NOs 4, 6, and 8, respectively;
(ix) FS22-172-003-AA/FS28-256-001 may be as set forth in SEQ ID NOs 53, 6, and 55, respectively;
(x) FS22-172-003-AA/FS28-256-005 may be as set forth in SEQ ID NOs 53, 6, and 55, respectively;
(xi) FS22-172-003-AA/FS28-256-014 may be as set forth in SEQ ID NOs 60, 6, and 62, respectively;
(xii) FS22-172-003-AA/FS28-256-018 may be as set forth in SEQ ID NOs 43, 6, and 45, respectively;
(xiii) FS22-172-003-AA/FS28-256 may be as set forth in SEQ ID NOs 67, 6, and 55, respectively;
(xiv) FS22-172-003-AA/FS28-024-051 may be as set forth in SEQ ID NOs 21, 23, and 72, respectively;
(xv) FS22-172-003-AA/FS28-024-053 may be as set forth in SEQ ID NOs 21, 23, and 77, respectively; and
(xvi) FS22-172-003-AA/FS28-024 may be as set forth in SEQ ID NOs 21, 23, 82, respectively;
wherein the CDR sequences are defined according to the IMGT numbering scheme.

The sequence of the VL domain CDR1, CDR2 and CDR3 of:
(i) FS22-172-003-AA/FS28-256-271 may be as set forth in SEQ ID NOs 12, 14, and 16, respectively;
(ii) FS22-172-003-AA/FS28-024-052 may be as set forth in SEQ ID NOs 12, 14, and 28, respectively;
(iii) FS22-172-003-AA/FS28-256-021 may be as set forth in SEQ ID NOs 12, 14, and 34, respectively;
(iv) FS22-172-003-AA/FS28-256-012 may be as set forth in SEQ ID NOs 12, 14, and 39, respectively;
(v) FS22-172-003-AA/FS28-256-023 may be as set forth in SEQ ID NOs 12, 14, and 34, respectively;
(vi) FS22-172-003-AA/FS28-256-024 may be as set forth in SEQ ID NOs 12, 14, and 49, respectively;
(vii) FS22-172-003-AA/FS28-256-026 may be as set forth in SEQ ID NOs 12, 14, and 49, respectively;
(viii) FS22-172-003-AA/FS28-256-027 may be as set forth in SEQ ID NOs 12, 14, and 16, respectively;
(ix) FS22-172-003-AA/FS28-256-001 may be as set forth in SEQ ID NOs 12, 14, and 34, respectively;
(x) FS22-172-003-AA/FS28-256-005 may be as set forth in SEQ ID NOs 12, 14, and 49, respectively;
(xi) FS22-172-003-AA/FS28-256-014 may be as set forth in SEQ ID NOs 12, 14, and 39, respectively;
(xii) FS22-172-003-AA/FS28-256-018 may be as set forth in SEQ ID NOs 12, 14, and 39, respectively;
(xiii) FS22-172-003-AA/FS28-256 may be as set forth in SEQ ID NOs 12, 14, and 39, respectively;
(xiv) FS22-172-003-AA/FS28-024-051 may be as set forth in SEQ ID NOs 12, 14, and 28, respectively;
(xv) FS22-172-003-AA/FS28-024-053 may be as set forth in SEQ ID NOs 12, 14 and 28, respectively; and
(xvi) FS22-172-003-AA/FS28-024 may be as set forth in SEQ ID NOs 12, 14 and 28, respectively;
wherein the CDR sequences are defined according to the IMGT numbering scheme.

For example, the sequence of the VH domain CDR1, CDR2 and CDR3 of:
(i) FS22-172-003-AA/FS28-256-271 may be as set forth in SEQ ID NOs 5, 7, and 9, respectively;
(ii) FS22-172-003-AA/FS28-024-052 may be as set forth in SEQ ID NOs 21, 23, and 25, respectively;
(iii) FS22-172-003-AA/FS28-256-021 may be as set forth in SEQ ID NOs 5, 31 and 9, respectively;
(iv) FS22-172-003-AA/FS28-256-012 may be as set forth in SEQ ID NOs 5, 31, and 9, respectively;
(v) FS22-172-003-AA/FS28-256-023 may be as set forth in SEQ ID NOs 44, 31, and 46, respectively;
(vi) FS22-172-003-AA/FS28-256-024 may be as set forth in SEQ ID NOs 5, 31, and 9, respectively;
(vii) FS22-172-003-AA/FS28-256-026 may be as set forth in SEQ ID NOs 44, 31, and 46, respectively;
(viii) FS22-172-003-AA/FS28-256-027 may be as set forth in SEQ ID NOs 5, 31, and 9, respectively;
(ix) FS22-172-003-AA/FS28-256-001 may be as set forth in SEQ ID NOs 54, 31, and 56, respectively;
(x) FS22-172-003-AA/FS28-256-005 may be as set forth in SEQ ID NOs 54, 31, and 56, respectively;
(xi) FS22-172-003-AA/FS28-256-014 may be as set forth in SEQ ID NOs 61, 31, and 63, respectively;
(xii) FS22-172-003-AA/FS28-256-018 may be as set forth in SEQ ID NOs 44, 31, and 46, respectively;
(xiii) FS22-172-003-AA/FS28-256 may be as set forth in SEQ ID NOs 68, 31, and 56, respectively;
(xiv) FS22-172-003-AA/FS28-024-051 may be as set forth in SEQ ID NOs 22, 24, and 73, respectively;
(xv) FS22-172-003-AA/FS28-024-053 may be as set forth in SEQ ID NOs 22, 24, and 78, respectively; and
(xvi) FS22-172-003-AA/FS28-024 may be as set forth in SEQ ID NOs 22, 24, and 83, respectively;
wherein the CDR sequences are defined according to the Kabat numbering scheme.

The sequence of the VL domain CDR1, CDR2 and CDR3 of:
(i) FS22-172-003-AA/FS28-256-271 may be as set forth in SEQ ID NOs 13, 15, and 16, respectively;
(ii) FS22-172-003-AA/FS28-024-052 may be as set forth in SEQ ID NOs 13, 15, and 28, respectively;
(iii) FS22-172-003-AA/FS28-256-021 may be as set forth in SEQ ID NOs 13, 15, and 34, respectively;
(iv) FS22-172-003-AA/FS28-256-012 may be as set forth in SEQ ID NOs 13, 15, and 39, respectively;
(v) FS22-172-003-AA/FS28-256-023 may be as set forth in SEQ ID NOs 13, 15, and 34, respectively;
(vi) FS22-172-003-AA/FS28-256-024 may be as set forth in SEQ ID NOs 13, 15, and 49, respectively;
(vii) FS22-172-003-AA/FS28-256-026 may be as set forth in SEQ ID NOs 13, 15, and 49, respectively;
(viii) FS22-172-003-AA/FS28-256-027 may be as set forth in SEQ ID NOs 13, 15, and 16, respectively;
(ix) FS22-172-003-AA/FS28-256-001 may be as set forth in SEQ ID NOs 13, 15, and 34, respectively;
(x) FS22-172-003-AA/FS28-256-005 may be as set forth in SEQ ID NOs 13, 15, and 49, respectively;
(xi) FS22-172-003-AA/FS28-256-014 may be as set forth in SEQ ID NOs 13, 15, and 39, respectively;
(xii) FS22-172-003-AA/FS28-256-018 may be as set forth in SEQ ID NOs 13, 15, and 39, respectively;
(xiii) FS22-172-003-AA/FS28-256 may be as set forth in SEQ ID NOs 13, 15, and 39, respectively;
(xiv) FS22-172-003-AA/FS28-024-051 may be as set forth in SEQ ID NOs 13, 15, and 28, respectively;
(xv) FS22-172-003-AA/FS28-024-053 may be as set forth in SEQ ID NOs 13, 15, and 28, respectively; and
(xvi) FS22-172-003-AA/FS28-024 may be as set forth in SEQ ID NOs 13, 15, and 28, respectively;
wherein the CDR sequences are defined according to the Kabat numbering scheme.

The CDR-based antigen-binding site may comprise the VH or VL domains, preferably the VH and VL domains, of antibody of antibody FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024,FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, or FS22-172-003-AA/FS28-024, preferably antibody FS22-172-003-AA/FS28-256-271 or FS22-172-003-AA/FS28-024-052, most preferably antibody FS22-172-003-AA/FS28-256-271.

The VH domain of antibodies FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, and FS22-172-003-AA/FS28-024 may have the sequence set forth in SEQ ID NOs 3, 19, 3, 3, 42, 3, 42, 3, 52, 52, 59, 42, 66, 71, 76, and 81, respectively.

The VL domain of antibodies FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, and FS22-172-003-AA/FS28-024 may have the sequence set forth in SEQ ID NOs 11, 27, 33, 38, 33, 48, 48, 11, 33, 48, 38, 38, 38, 27, 27, and 27, respectively.

The bispecific antibody molecule of the invention comprises a CD137 antigen-binding site. The CD137 antigen-binding site is located in a constant domain of the antibody molecule, preferably a CH3 domain. The CD137 antigen-binding site comprises one or more modified structural loops in a constant domain of the antibody molecule. Engineering antibody constant domain structural loops to create antigen-binding sites for target antigens is known in the art and is described, for example, Wozniak-Knopp G et al. (2010) Protein Eng Des. 23 (4): 289-297; WO2006/072620 and WO2009/132876. The CD137 constant domain antigen-binding site comprised in the antibody molecules of the invention was identified following an extensive selection and affinity maturation program, and preferentially binds to dimeric rather than monomeric human CD137.

The CD137 antigen-binding site of the bispecific antibody molecule comprises a first and second sequence, wherein the first and second sequences are located in the AB and EF structural loops of the constant domain, preferably the CH3 domain, of the bispecific antibody molecule, respectively. The first sequence and second sequence are preferably the first and second sequence of FS22-172-003 set forth in SEQ ID NOs 87 and 88, respectively. The first and second sequences are preferably located between positions 14 and 17, and positions 91 and 99, of the CH3 domain of the bispecific antibody molecule, respectively, wherein the residue numbering is according to IMGT numbering. The CD loop sequence of the bispecific antibody molecule is preferably unmodified, i.e. wild-type.

The CD loop sequence therefore preferably has the sequence set forth in SEQ ID NO: 89. The CD loop sequence is preferably located at positions 43 to 78 of the CH3 domain of the bispecific antibody molecule, wherein the residue numbering is according to IMGT numbering.

In a preferred embodiment, the bispecific antibody molecule comprises a CH3 domain which comprises, has, or consists of the CH3 domain sequence of FS22-172-003 set forth in SEQ ID NO: 86. The CH3 domain of the bispecific antibody molecule may optionally comprise an additional lysine residue (K) at the immediate C-terminus of the CH3 domain sequence.

In an preferred embodiment, the bispecific antibody molecule comprises the heavy chain and/or light chain, preferably the heavy chain and light chain, of antibody:
(i) FS22-172-003-AA/FS28-256-271 set forth in SEQ ID NOs 2 and 10, respectively;
(ii) FS22-172-003-AA/FS28-024-052 set forth in SEQ ID NOs 18 and 26, respectively;
(iii) FS22-172-003-AA/FS28-256-021 set forth in SEQ ID NOs 30 and 32, respectively;
(iv) FS22-172-003-AA/FS28-256-012 set forth in SEQ ID NOs 36 and 37, respectively;
(v) FS22-172-003-AA/FS28-256-023 set forth in SEQ ID NOs 41 and 32, respectively;
(vi) FS22-172-003-AA/FS28-256-024 set forth in SEQ ID NOs 30 and 47, respectively;
(vii) FS22-172-003-AA/FS28-256-026 set forth in SEQ ID NOs 41 and 47, respectively;
(viii) FS22-172-003-AA/FS28-256-027 set forth in SEQ ID NOs 30 and 10, respectively;
(ix) FS22-172-003-AA/FS28-256-001 set forth in SEQ ID NOs 51 and 32, respectively;
(x) FS22-172-003-AA/FS28-256-005 set forth in SEQ ID NOs 51 and 47, respectively;
(xi) FS22-172-003-AA/FS28-256-014 set forth in SEQ ID NOs 58 and 37, respectively;
(xii) FS22-172-003-AA/FS28-256-018 set forth in SEQ ID NOs 41 and 37, respectively;
(xiii) FS22-172-003-AA/FS28-256 set forth in SEQ ID NOs 65 and 37, respectively;
(xiv) FS22-172-003-AA/FS28-024-051 set forth in SEQ ID NOs 70 and 26, respectively;
(xv) FS22-172-003-AA/FS28-024-053 set forth in SEQ ID NOs 75 and 26, respectively;
or
(xvi) FS22-172-003-AA/FS28-024 set forth in SEQ ID NOs 80 and 26, respectively.

In a more preferred embodiment, the bispecific antibody molecule comprises the heavy chain and/or light chain, preferably the heavy chain and light chain, of: antibody FS22-172-003-AA/FS28-256-271 or FS22-172-003-AA/FS28-024-052, most preferably antibody FS22-172-003-AA/FS28-256-271, wherein the heavy and light chain sequences of these antibodies are as set out above.

The bispecific antibody molecules of the present invention may also comprise variants of a first, second or third sequence, AB, CD or EF structural loop sequence, CH3 domain, CH2 domain, CDR, VH domain, VL domain, light chain and/or heavy chain sequences disclosed herein. Suitable variants can be obtained by means of methods of sequence alteration, or mutation, and screening. In a preferred embodiment, an antibody molecule comprising one or more variant sequences retains one or more of the functional characteristics of the parent antibody molecule, such as binding specificity and/or binding affinity for MSLN and CD137. For example, an antibody molecule comprising one or more variant sequences preferably binds to MSLN and/or CD137 with the same affinity, or a higher affinity, than the (parent) antibody molecule. The parent antibody molecule is an antibody molecule which does not comprise the amino acid substitution(s), deletion(s), and/or insertion(s) which have been incorporated into the variant antibody molecule.

An antibody molecule which comprises CDRs 1-6, the VH domain, and/or the heavy chain of antibody FS22-172-003-AA/FS28-256-271, FS22-172-003-AA/FS28-024-052 FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, or FS22-172-003-AA/FS28-024 may comprise an amino acid substitution at position 55 or 57 of the VH domain, wherein the amino acid residue numbering is according to the IMGT numbering scheme.

For example, the antibody molecule may comprise CDRs 1-6, the VH domain, and/or the heavy chain of antibody FS22-172-003-AA/FS28-256-027, wherein the antibody molecule comprises an amino acid substitution at position 55 of the VH domain, and wherein the amino acid residue numbering is according to the IMGT numbering scheme. For example, an antibody molecule of the invention may comprise a first, second or third sequence, AB, CD or EF structural loop sequence, CH3 domain, CH2 domain, CDR, VH domain, VL domain, light chain and/or heavy chain sequence which has at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a structural loop, CH3 domain, CH2 domain, CDR, VH domain, VL domain, light chain or heavy chain sequence disclosed herein.

In a preferred embodiment, the bispecific antibody molecule of the invention comprises a CH3 domain sequence which has at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a CH3 domain as disclosed herein.

In a further preferred embodiment, the bispecific antibody molecule has or comprises a CH2 domain sequence, which has at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a CH2 domain as disclosed herein.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used, e.g. BLAST (which uses the method of Altschul et al., 1990), FASTA (which uses the method of Pearson and Lipman, 1988), or the Smith-Waterman algorithm (Smith and Waterman, 1981), or the TBLASTN program, of Altschul et al., 1990 supra, generally employing default parameters. In particular, the psi-Blast algorithm (Altschul et al., 1997) may be used.

The bispecific antibody molecule of the invention may also comprise a first, second or third sequence, AB, CD or EF structural loop sequence, CH3 domain, CH2 domain, VH domain, VL domain, light chain and/or heavy chain which has one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with a first, second or third sequence, AB, CD or EF structural loop sequence, CH3 domain, CH2 domain, Fcab, CDR, VH domain, VL domain, light chain or heavy chain sequence disclosed herein. In particular, alterations may be made in one or more framework regions of the antibody molecule outside the VH and VL domain sequences and/or in one or more framework regions of the CH3 domain. For example, the alterations may be in the CH3 domain outside of the sequences described herein as a first, second and third sequences, or as AB, CD or EF structural loop sequences.

The bispecific antibody molecule may comprise a VH CDR1, VH CDR2, VH CDR3, VL CDR1, VL CDR2, and/or VL CDR3 which has one or more amino acid sequence alterations (addition, deletion, substitution and/or insertion of an amino acid residue), preferably 3 alterations or fewer, 2 alterations or fewer, or 1 alteration compared with the VH CDR1, VH CDR2, VH CDR3, VL CDR1, VL CDR2, and/or VL CDR3 as disclosed herein.

In one embodiment, the bispecific antibody molecule of the invention comprises a CH3 domain sequence with one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the CH3 domain as disclosed herein.

In embodiments in which one or more amino acids are substituted with another amino acid, the substitutions may conservative substitutions, for example according to the following Table. In some embodiments, amino acids in the same category in the middle column are substituted for one another, i.e. a non-polar amino acid is substituted with another non-polar amino acid, for example. In some embodiments, amino acids in the same line in the rightmost column are substituted for one another.

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g. binding affinity) of the antibody molecule comprising the substitution as compared to the equivalent unsubstituted antibody molecule.

The word “tumor” refers to a mass of cells of abnormal size and/or composition resulting from increased proliferation and/or prolonged survival of cells. Tumors may be benign or malign. In the latter case they are referred to as “cancer”. A “tumor” cell therefore is a cell which possesses an abnormally increased ability to divide and/or to resist cell death compared with other cells of the same cell type.

Cancer is characterised by the abnormal proliferation of malignant tumor cells. Where a particular type of cancer, such as ovarian cancer, is referred to, this refers to an abnormal proliferation of malignant cells of the relevant tissue, such as breast tissue. A secondary or metastatic cancer which is located in the breast but is the result of abnormal proliferation of malignant cells of another tissue, such as ovarian tissue, is not a breast cancer as referred to herein but an ovarian cancer.

MSLN is expressed on the surface of some tumor cells and high expression levels of soluble MSLN have been correlated with poor prognosis in several cancers. Anti-MSLN antibodies have been investigated as anti-cancer therapeutics. These anti-MSLN antibodies either induce direct cell killing through their ADCC activity or are used in the form of ADCs.

Accordingly, the cancer to be treated using a bispecific antibody molecule that binds MSLN and CD137 in combination with a PD-1/PD-L1 inhibitor therefore preferably expresses, or has been determined to express, MSLN. More preferably, cells of the cancer to be treated comprise, or have been determined to comprise, MSLN at their cell surface, i.e. to comprise cell-surface bound MSLN.

The cancer preferably comprises, or has been determined to comprise, tumor infiltrating lymphocytes (TILs) that express CD137. Specifically, the TILs preferably comprise, or have been determined to comprise, CD137 on their cell surface.

The cancer may be a primary or a secondary cancer. Thus, an antibody molecule that binds MSLN and CD137 as described herein may be for use in a method of treating cancer in an individual in combination with a PD-1/PD-L1 inhibitor, wherein the cancer is a primary tumour and/or a secondary or metastatic tumor.

The cancer to be treated may be a solid cancer.

As mentioned above, the cancer to be treated may be a cancer that expresses MSLN or has been determined to express MSLN. Preferably, the cancer is selected from the group consisting of: ovarian cancer, pancreatic adenocarcinoma, mesothelioma, cervical carcinoma and non-small cell lung cancer.

The patient to be treated may be selected for treatment if the cancer expresses MSLN. The patient may be selected if the cancer has been determined to express MSLN. Preferably, the patient is selected for treatment if the cancer is any one of ovarian cancer, pancreatic adenocarcinoma, mesothelioma, cervical carcinoma or non-small cell lung cancer and expressed MSLN.

The present inventors showed that combined treatment with M9657 (SEQ ID NO: 2 and SEQ ID NO: 10) and pembrolizumab (SEQ ID NO: 93 and SEQ ID NO: 94) resulted in a greater increase in T cell activation, tumor target cell killing by CD8+ cells and cytokine release than the combined increase in T cell activation, tumor target cell killing by CD8+ cells and cytokine release from monotherapy with either M9657 or pembrolizumab. The present inventors also showed that combination therapy of FS122m and anti-mPD-1 resulted in greater tumor growth retardation, tumor volume reduction, median survival increase and/or increase in the number of complete tumor regressions than the combined tumor growth retardation, tumor volume reduction, median survival increase and/or increase in the number of complete tumor regressions observed when mice were treated with either FS122m or anti-mPD-1 alone. Accordingly, the present inventors found that combined treatment with M9657 and pembrolizumab increased T cell activation, tumor target cell killing by CD8+ cells and cytokine release in a synergistic manner. Similarly, combined treatment with F122m (SEQ ID NO: 84 and SEQ ID NO: 85) and anti-mPD-1 retarded tumor growth, reduced tumor volume, increased median survival and increased the number of complete tumor regressions in a synergistic manner.

In one embodiment, the antibody molecule that binds MSLN and CD137 in combination with a PD-1/PD-L1 inhibitor results in tumor growth retardation, tumor volume reduction, median survival increase and/or increase in the number of complete tumor regressions. Preferably, the antibody molecule that binds MSLN and CD137 in combination with a PD-1/PD-L1 inhibitor results in statistically significantly greater tumor growth retardation, tumor volume reduction, median survival increase and/or increase in the number of complete tumor regressions than monotherapy treatment with the antibody molecule that binds MSLN and CD137 or monotherapy treatment with the PD-1/PD-L1 inhibitor. More preferably, the antibody molecule that binds MSLN and CD137 in combination with a PD-1/PD-L1 inhibitor results in greater tumor growth retardation, tumor volume reduction, median survival increase and/or increases in the number of complete tumor regressions than the combined tumor growth retardation, tumor volume reduction, median survival increase and/or increase in the number of complete tumor regressions of monotherapy treatment with the antibody molecule that binds MSLN and CD137 and monotherapy treatment with the PD-1/PD-L1 inhibitor.

In another embodiment, the antibody molecule that binds MSLN and CD137 in combination with a PD-1/PD-L1 inhibitor increases T cell activation, tumor target cell killing by CD8+ cells and cytokine release. Preferably, the antibody molecule that binds MSLN and CD137 in combination with a PD-1/PD-L1 inhibitor results in statistically significantly greater T cell activation, tumor target cell killing by CD8+ cells and cytokine release than monotherapy with either the bispecific antibody molecule that binds MSLN and CD137 or the PD-1/PD-L1 inhibitor. More preferably, the antibody molecule that binds MSLN and CD137 in combination with the PD-1/PD-L1 inhibitor results in greater T cell activation, tumor target cell killing by CD8+ cells and cytokine release than the combined T cell activation, tumor target cell killing by CD8+ cells and cytokine release of monotherapy treatment with the antibody molecule that binds MSLN and CD137 and monotherapy treatment with the PD-1/PD-L1 inhibitor.

The ability of the antibody molecule that binds MSLN and CD137 in combination with the PD-1/PD-L1 inhibitor to activate T cells may be determined by measuring the maximum bioluminescence signal transmitted by T cells in a with Bio-Glo™-NL Luciferase Assay in the presence of the antibody molecule that binds MSLN and CD137 in combination with the PD-1/PD-L1 inhibitor.

The ability of the antibody molecule that binds MSLN and CD137 in combination with a PD-1/PD-L1 inhibitor to activate T cells may also be determined by measuring IFNγ release in a Cytokine Release Assay in the presence of the bispecific antibody molecule in combination with the PD-1/PD-L1 inhibitor.

In one embodiment, the antibody molecule that binds MSLN and CD137 and PD-1/PD-L1 inhibitor are administered as a first (“front”) line of treatment (e.g., the initial or first treatment). In another embodiment, the antibody molecule that binds MSLN and CD137 and the PD-1/PD-L1 inhibitor are administered as a second line of treatment (e.g., after initial treatment with the same or a different therapeutic, including after relapse and/or where the first treatment has failed).

“Administering” as used herein refers to the physical introduction of a therapeutic agent to a subject, using any of the various methods and delivery systems known to those skilled in the art. Exemplary routes of administration for the antibody molecule that binds MSLN and CD137 and/or the PD-1/PD-L1 inhibitor include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route. Non-parenteral routes include a topical, epidermal or mucosal route of administration, for example, orally, intranasally, vaginally, rectally, sublingually, or topically.

Accordingly, in some embodiments the antibody molecule that binds MSLN and CD137 and/or the PD-1/PD-L1 inhibitor are administered parenterally. The antibody molecule that binds MSLN and CD137 and/or the PD-1/PD-L1 inhibitor may be administered intravenously, intramuscularly, subcutaneously, intraperitoneally or spinally. Alternatively, the antibody molecule that binds MSLN and CD137 and/or the PD-1/PD-L1 inhibitor may be administered by injection or infusion.

In other embodiments, the embodiments the antibody molecule that binds MSLN and CD137 and/or the PD-1/PD-L1 inhibitor are administered non-parenterally. The antibody molecule that binds MSLN and CD137 and/or the PD-1/PD-L1 inhibitor may be administered orally, intranasally, vaginally, rectally, sublingually, or topically.

“Concomitant administration” describes the simultaneous administration of the antibody molecule that binds MSLN and CD137 and the PD-1/PD-L1 inhibitor in the same or in separate formulations. “Sequential administration” refers to the timely separated administration of the bispecific antibody molecule and the PD-1/PD-L1 inhibitor in separate formulations.

Thus, the antibody molecule that binds MSLN and CD137 and the PD-1/PD-L1 inhibitor may be part of the same formulation or part of separate formulations. Preferably, the antibody molecule that binds MSLN and CD137 and the PD-1/PD-L1 inhibitor are provided as separate formulations.

In one embodiment of the invention, the antibody molecule that binds MSLN and CD137 and the PD-1/PD-L1 inhibitor are administered concomitantly. For example, the bispecific antibody molecule that binds MSLN and CD137 may be administered with the PD-1/PD-L1 inhibitor in the same formulation. Alternatively, the antibody molecule that binds MSLN and CD137 and the PD-1/PD-L1 inhibitor may be administered in separate formulations immediately before or after one another.

Preferably, the antibody molecule that binds MSLN and CD137 and the PD-1/PD-L1 inhibitor are administered to the patient sequentially. More preferably, the antibody molecule that binds MSLN and CD137 and the PD-1/PD-L1 inhibitor are administered to the patient within 4 days of each other, preferably within 3 days of each other, more preferably within 2 days of each other, or sequentially on the same day.

The present invention also relates to a method of treating cancer comprising administering to the individual in need thereof an antibody molecule that binds MSLN and CD137 and a PD-1/PD-L1 inhibitor.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. Appropriate doses of antibody molecules are well known in the art (Ledermann et al., 1991; Bagshawe et al., 1991). A therapeutically effective amount or suitable dose of an antibody molecule can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antibody molecule.

Accordingly, the invention may relate to a method of treating cancer comprising administering to the individual in need thereof a therapeutically effective amount of an antibody molecule that binds MSLN and CD137 and a therapeutically effective amount of a PD-1/PD-L1 inhibitor.

Also provided is a kit comprising an antibody molecule that binds MSLN and CD137 and a PD-1/PD-L1 inhibitor. Preferably, the kit may comprise an antibody molecule that binds MSLN and CD137 and a pharmaceutically acceptable excipient and a PD-1/PD-L1 inhibitor and a pharmaceutically acceptable excipient.

The kit may be a package comprising a first container and a second container, the first container comprising the antibody molecule that binds MSLN and CD137, the second container comprising the PD-1/PD-L1 inhibitor. The package may comprise instructions for use of the antibody molecule that binds MSLN and CD137 in combination with the PD-1/PD-L1 inhibitor for the treatment of cancer in an individual.

In one embodiment, the kit may be a package comprising at least one dose of a medicament comprising the antibody molecule that binds MSLN and CD137 and one dose of a medicament comprising the PD-1/PD-L1 inhibitor. Preferably, the kit may comprise at least one dose of a medicament comprising the antibody molecule that binds MSLN and CD137 and a pharmaceutically acceptable excipient and one dose of a medicament comprising the PD-1/PD-L1 inhibitor and a pharmaceutically acceptable excipient. More preferably, the kit may further comprise a package insert comprising instructions for treating cancer in an individual using the medicaments.

In another embodiment, the kit may be a package comprising a first container and a second container, the first container comprising the antibody molecule that binds MSLN and CD137, the second container comprising the PD-1/PD-L1 inhibitor. The first container may comprise at least one dose of a medicament comprising the antibody molecule that binds MSLN and CD137 and a pharmaceutically acceptable excipient and the second container may compromise at least one dose of a medicament comprising the PD-1/PD-L1 inhibitor and a pharmaceutically acceptable excipient. The package may further comprise an insert comprising instructions for using the medicaments for the treatment of cancer in an individual.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Materials and Methods

The efficacy of the combination of an antibody molecule that binds MSLN and CD137 and a PD-1/PD-L1 inhibitor in enhancing an anti-cancer immune response compared with monotherapy with either the antibody molecule that binds MSLN and CD137 or the PD-1/PD-L1 inhibitor was assessed by studying different parameters. These include T cell activation, cytotoxicity of target cells and cytokine release in vitro in human cells or average tumour volume over time and prolonged survival in vivo in mouse tumour models. These methods are described in more detail below.

### Bio-Glo™-NL Luciferase Assay

In order to study the combined impact of PD-1 blocking and stimulating CD137 stimulation, the efficacy of combination treatment with M9657 and the pembrolizumab on NF-κB expression in PD-1+CD137 effector cells was measured in a Bio-Glo™-NL Luciferase Assay. The assay was performed according to the manufacturer's instructions. One day before the assay, PD-L1 aAPC/CHO-K1 cells expressing PD-L1 and a T cell activating agent were thawed and recovered in 14.5 mL cell recovery medium (10% FBS/F-12). Cells were then plated in a white 96-well plate and incubated overnight at 37 °C in a 5% CO₂ incubator. On the day of the assay, serial dilutions of antibodies were prepared of M9657 (250 nM serially diluted 4-fold to 0.003815 nM), pembrolizumab (167.875 nM), CD137L (250 nM serially diluted 4-fold to 0.003815 nM), and anti-HIS tagged antibody (125 nM serially diluted 4-fold to 0.0019 nM), the latter of which was added to CD137L for crosslinking. CD137 ligand (CD137L) interacts with CD137 to induce T cell activation. Here CD137L was used as positive control in the reporting assay. An antibody directed to an irrelevant target (anti-HEL-hIgG1-LALA antibody) was used as negative control. Media from the PD-L1 aAPC/CHO-K1 cells was aspirated, and a total of 40 μL of prepared antibody solution per well was added to each well. PD-1+CD137 effector cells were thawed and recovered, and 40 μL of cell suspension was added to each well. MSLN expressing CHO cells were added to the wells at 1:1 ratio of PD-L1 aAPC/CHO-K1 (75,000 cells): CHO-MSLN cells (75,000 cells). Plates were incubated for 6 hours and at the end of the incubation period Bio-Glo-NL luciferase reagent was prepared by combining 1 volume of Bio-Glo-NL Luciferase Assay Substrate with 50 volumes of Bio-Glo-NL Luciferase Assay Buffer. Assay plates were removed from the incubator, 80 μL of the Bio-Glo-NL reagent was added to each well, and the plates were incubated at room temperature for 5 minutes. Following incubation, the plates were read using Tecan plate reader with luminescence setting. GraphPad Prism V9 software was used for statistical analysis and for plotting graphs. To calculate the efficacy of combination treatment with an antibody molecule that binds MSLN and CD137 and a PD-1/PD-L1 inhibitor to induce luciferase activity compared with monotherapy with the antibody molecule that binds MSLN and CD137 or the PD-1/PD-L1 inhibitor, the antibody concentrations at which the half maximal effective concentration (EC₅₀) is achieved were calculated using non-linear regression fit.

### Target Dependent Cytotoxicity and Cytokine Release Assays

The efficacy of combined PD-1 blocking and CD137 stimulation on tumor target cell killing and cytokine release by CD8+ T cells was also studied. To this end, toxicity of CD8+ T cells against co-cultured NCI-H226 target cancer cells and IFNγ release were monitored ex vivo. NCI-H226 cells were cultured in the RPMI-1640 medium with 10% FBS. Human CD8⁺ T cells were purchased from Hemacare. All cells were cultured at 37 °C, 5% CO2, and 95% relative humidity. NCI-H226 cells were treated with 10 ng/ml of human IFNγ for 48 hours, washed and labeled with IncuCyte Cytolight Rapid Red dye one day before the experiment, and then seeded in assay plates. Human CD8⁺ T cells were thawed and recovered in 250 U/ml IL-2 for 24 hours and then added to the assay plates on the day of the experiment. (NCI-H226 cells express a high level of MSLN and EGFR in the presence or absence of 2 pM anti-CD3×anti-EGFR BiTE. Subsequently, fixed concentrations of 2 pM anti-CD3×anti-EGFR BiTE, 10 μg/ml of pembrolizumab and 1 μM Cytolight Rapid Dye and serial dilutions of M9657 or control antibodies were added to the cells and incubated for 72 hours. Cell growth was detected via IncuCyte@ live-imaging system. Cytotoxicity of target cells was detected via an Incucyte@ Live-Cell Analysis System and determined by calculating % killing over the BiTE only group. Both total cells and labelled cells were monitored using the phase contrast and red fluorescence channel, respectively. IFNγ release was detected by AlphaLisa following the manufacturer's instructions.

### Mouse Tumour Models

Wild-type BALB/c and C57BL/6 female mice were purchased from Charles River Laboratories, or from Lingchang Biological Technology Co. LTD. All mice were 8-12 weeks old at the start of studies and were housed and maintained as described below, under conditions that conform to The Guide for the Care and Use of Laboratory Animals, 8th Edition. All animal experiments were performed in accordance with EMD Serono Research Institutional (protocol 17-008, 20-005) and Wuxi AppTec Animal Care and Use Committee (IACUC) guidelines.

Upon arrival at the research institute's vivarium, all animals received a detailed physical examination, including body weight measurement, by the research staff. All animals were found to be in satisfactory health. Animals were housed in a specific pathogen free barrier animal facility at EMD Serono. The mice were kept in individual ventilation cages at constant temperature and humidity with 5 animals in each cage. The identification labels for each cage contained the following information: number of animals, sex, strain, date received, treatment, study number, group number and the starting date of the treatment. Animals were marked by ear notches or ear tags. Animal holding rooms were maintained at 20-26 °C and 40-70% humidity. Lights were on a 12-hour light/dark cycle. Animals had free access to a standard certified commercial laboratory diet. Maximum allowable concentrations of contaminants in the diet were controlled and routinely analysed by the manufacturers. Autoclaved municipal tap water, suitable for human consumption was available to the animals ad libitum. It is considered that there are no known contaminants in the dietary materials that could influence the tumor growth. A period of approximately one week was allowed between animal arrival and tumor inoculation in order for the animals to acclimatize to the laboratory environment. All the procedures related to animal handling, care, and treatment in the study were performed according to the guidelines approved by the institutional animal care and use committees (IACUCs) of EMD Serono and following the guidance of the Association for Assessment and Accreditation of Laboratory Animal Care (AAALAC).

E0771, JC and Eph4-1424 tumor models were used to investigate the antitumor effect of a combination of an antibody molecule that binds MSLN and CD137 and a PD-1/PD-L1 inhibitor compared with monotherapy with the antibody molecule that binds MSLN and CD137 or the PD-1/PD-L1 inhibitor. Since M9657 does not show cross-reactivity for mouse MSLN and mouse CD137, anti-mMSLN-mCD137-huIgG1-LALA (FS122m) (SEQ ID NO: 84 and 85) was developed as a surrogate antibody of M9657 for in vivo pharmacology studies. For the E0771 model, female C57BL/6 mice were inoculated orthotopically in the right mammary fat pad with 0.5×106 E0771 tumor cells in 0.1 mL of PBS. Mice were randomly assigned to treatment groups (n=9 mice/group) when the average tumor volume reached approximately 50-100 mm³. For the JC model, female BALB/c mice were inoculated subcutaneously (sc) at the right upper flank with JC tumor cells (5×106) in 0.1 mL of PBS. Mice were randomly assigned to treatment groups (n=10 mice/group) when the average tumor volume reached approximately 50-100 mm³. For the Eph4-1424 model, female BALB/c mice were inoculated subcutaneously (sc) at the right upper flank with Eph4-1424 tumor cells (1×106) in 0.1 mL of PBS. Mice were randomly assigned to treatment groups (n=10 mice/group) when the average tumor volume reached approximately 50-100 mm³.

Mortality checks were performed once daily. Animals were checked daily for any effects of tumor growth and treatments on normal behavior such as mobility, food and water consumption (visual inspection only), eye/hair matting and any other abnormal effect as stated in the protocol. Body weights were recorded twice weekly and any mouse with a 20% loss of initial body weight was humanely sacrificed. Mice were humanely euthanized if their subcutaneous tumors reached a volume of 2500 mm³.

If tumor ulceration occurred, animals with ulcerated tumors were monitored at least 3 times per week with increasing frequency, up to once daily, depending upon clinical signs. Ulcerated tumors that did not scab over were cleaned with an appropriate wound cleansing solution (e.g., Novalsan). Antibiotic cream was applied to the ulcer/lesion only if directed by the veterinary staff.

Criteria for euthanasia included the lesion not healing or forming a scab within 1 week, the lesion being greater than 5 mm diameter, the lesion becoming cavitated or developing signs of infection (such as presence of pus) or bleeding, or the animal showing signs of discomfort (e.g. excessive licking and biting directed at the site) or systemic signs of illness (lethargy, decreased activity, decreased food consumption, decreased body condition or weight loss). The veterinary staff were consulted to discuss any possible exceptions.

Animals were euthanized if they were found to be moribund. Clinical examples of morbidity may include the animal being hunched, persistent recumbency and lack of response to handling or other stimuli, signs of severe organ or system failure, emaciation, hypothermia, CNS deficits (convulsions), respiratory symptoms (rapid respiratory rate, labored breathing, coughing, rales), gastrointestinal symptoms (diarrhea lasting >2 days, jaundice). Any animal that exhibited the above clinical issues was humanely sacrificed by CO₂.

Body weight was measured and recorded twice weekly until an endpoint was reached. Tumor sizes were measured twice per week in three dimensions using a caliper, and the volume was expressed in mm³ using the formula: width×length×height×0.5236.

Differences in tumor growth between treatment groups were determined using two-way analysis of variance (ANOVA) and Tukey's Multiple Comparisons Test. The significance of survival was determined using Log-rank (Mantel-Cox) test. All analyses were conducted using the GraphPad Prism software package (Prism 5 for Windows, Version 8.0, GraphPad Software Inc., San Diego, CA) and statistical significance was accepted at the p≤0.05 level.

### Example 1: NF-κB Expression

Activation of NF-κB expression is an indicator of T cell activation. CD137 clustering activates the NF-κB signalling pathway when it interacts with its cognate ligand, CD137L. Agonist antibody molecules mimic the ligand in driving clustering and activation of CD137, thereby activating the NF-κB signalling pathway. Anti-PD-1/PD-L1 antibodies on the other hand limit negative effects of PD-1 signalling on T cell activation by abrogating the interaction of PD-L1 with PD-1. NF-κB expression is measured as a function of luciferase activity resulting in a bioluminescence signal in the luciferase reporter assay described in Materials and Methods. Expression of luciferase in the assay is controlled by an NF-κB-sensitive promoter and therefore directly correlated with NF-κB expression.

In order to study the combined impact of PD-1 blocking and CD137 stimulation, the effect of combined treatment with M9657 and pembrolizumab was measured using a Bio-Glo™-NL Luciferase Assay. PD-1+CD137 effector cells were co-cultured with PD-L1aAPC/CHO K1 cells and treated with either of M9657+pembrolizumab, M9657, M9657+Anti-HEL-hIgG1-LALA, CD137L+Anti-HEL-hIgG1-LALA, CD137L+pembrolizumab, or CD137L+Anti-HEL-hIgG1-LALA. Since the bispecific antibody molecule M9657 requires binding to MSLN for its bioactivity and PD-L1 aAPC/CHO-K1 cells do not express MSLN, MSLN expressing CHO cells were added to the PD-L1 aAPC/CHO-K1 and CHO-MSLN cells. Luciferase activity was measured as a function of bioluminescence using a Tecan device and analysed using GraphPad Prism V9 software.

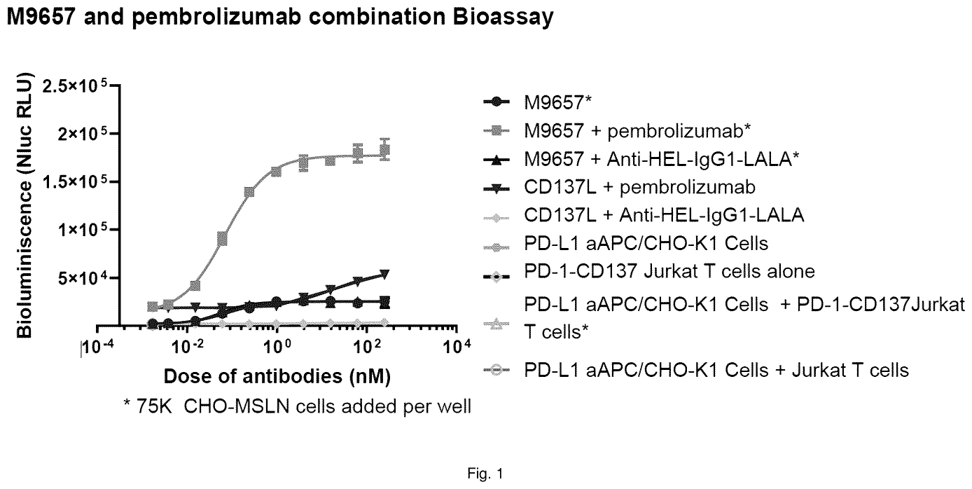

Treatment with M9657 triggered a concentration-dependent bioluminescence signal, enhancing T cell activation with an EC₅₀ of 0.08024 nM. Combined treatment with M9567+pembrolizumab increased the bioluminescence signal compared to treatment with any of M9657, M9657+Anti-HEL-hIgG1-LALA, CD137L+Anti-HEL-hIgG1-LALA, CD137L+pembrolizumab, and CD137L+Anti-HEL-hIgG1-LALA. Pembrolizumab showed an EC₅₀ of 0.06069 nM (FIG. 1).

### Example 2: Cytotoxicity and Cytokine Release

Two further hallmarks of T cell activation are human CD8⁺ T cell-mediated tumor target cell killing and cytokine release. To this end, NCI-H226 cancer cells were co-cultured ex vivo with CD8⁺ T cells to study the effect of combining CD137 stimulation and PD-1 blocking (Materials and Methods). Tumour target cell killing was measured as % killing of NCI-H226 cancer cells while cytokine release by CD8⁺ T cells was measured as INFγ levels in T cell culture supernatants.

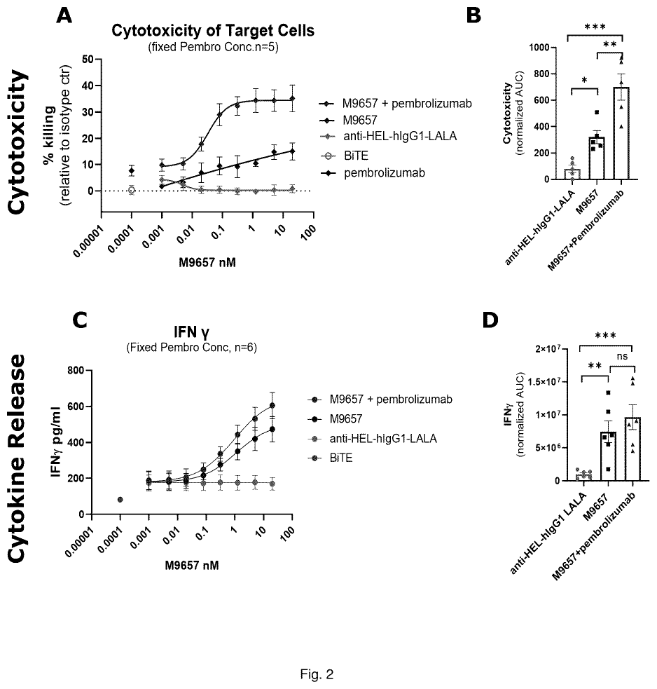

To calculate the effect of treating the co-cultured cells with M9657+pembrolizumab, M9657, isotype control (anti-HEL-hIgG1-LALA antibody), or BiTE (anti-CD3×anti-EGFR BiTE), cytotoxicity and INFγ release were measured. Treatment with M9657 resulted in concentration-dependent tumor target cell killing and INFγ release, which was further increased in cells treated with a combination of M9657 and pembrolizumab (FIG. 2). CD8⁺ T cell killing of target cells was elevated at all concentrations of M9657 with pembrolizumab tested compared with M9657 and isotype control monotherapy and rose particularly sharply between a concentration of 10 and 100 μM (FIG. 2A).

### Example 3: In Vivo Proof of Concept

Having shown that the combination of a CD137/MSLN mAb² with an anti-PD-1/PD-L1 antibody results in improved T cell activation, killing of target cells and T cell cytokine release, the effect of this combination was also tested in an in vivo mouse tumor mouse model.

Due to the lack of cross reactivity between M9657 and murine CD137, an anti-mMSLN-mCD137-huIgG1-LALA (FS112m) bispecific antibody having a similar binding affinity for murine CD137 as that of M9657 for human CD137 was developed as a surrogate antibody for use in mouse tumor models. FS112m was combined with an anti-mPD-1 to test the anti-tumor efficacy of both antibodies in combination compared with FS112m and anti-mPD-1 monotherapy in three different mouse breast tumor models. To this end, female mice were inoculated with either E0771, JC or Eph4-1424 breast cancer cells and randomly assigned to treatment groups when the average tumor volume reached approximately 50-100 mm³. Mice were inoculated, treated and treatment terminated according to the method described in Materials and Methods.

In all three mouse breast tumor models, the combination of FS112m with anti-mPD-1 was compared with FS112m and anti-mPD-1 monotherapy and monotherapy with an anti-HEL-hIgG1-LALA isotype control antibody. The results are outlined below.

### 3.1 E0771 Mouse Breast Tumor Model

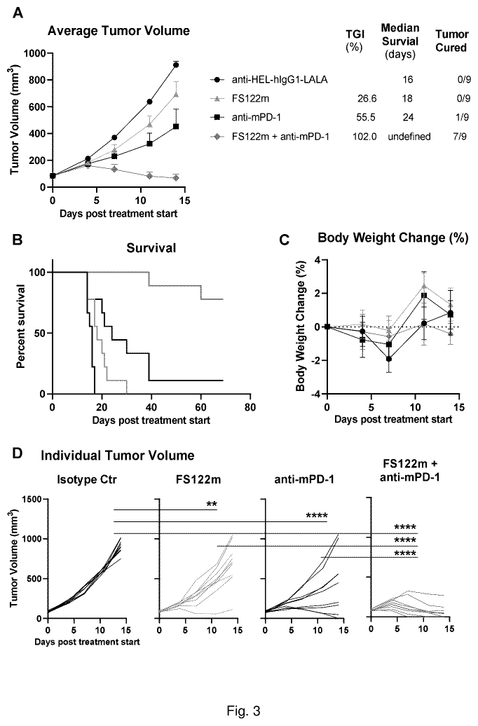

In the E0771 mouse breast tumor model, FS122m and anti-mPD-1 monotherapies induced marginal or moderate tumor growth inhibition (TGI) (26.6% and 55.5%, respectively) relative to the isotype control (P<0.01, P<0.0001, respectively, Day 14), and prolonged median survival (18 and 24 days, respectively) relative to the isotype control (16 days) (FIGS. 3A, B and D). TGI was further enhanced when mice were treated with a combination of FS122m and anti-mPD1 (102%) relative to FS122m (P<0.0001, Day 14) and anti-mPD1 (P<0.0001, Day 14) monotherapy (FIG. 3A). Combined FS122m and anti-mPD-1 treatment also prolonged median survival (FIG. 3B) and induced complete tumor regression in 7 out of 9 mice, compared with no complete tumor regression in 9 mice treated with FS122m monotherapy and complete tumor regression in only 1 of 9 mice treated with anti-mPD-1 monotherapy (FIG. 3D). Mice in the monotherapy and combination treatment groups showed no additional mortality or clinical signs, and body weight (BW) changes were comparable to those in the isotype control group, demonstrating that all of the treatments were well tolerated (FIG. 3C).

### 3.2 JC Mouse Breast Tumor Model

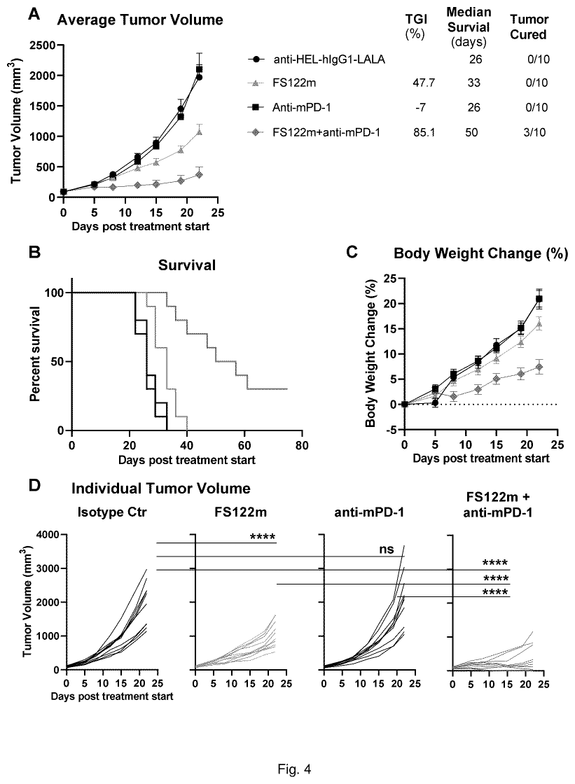

In the JC mouse breast tumor model, anti-mPD1 monotherapy did not show any anti-tumor effect (TGI=7%), while FS122m monotherapy displayed marginal TGI (47.7%) relative to the isotype control (P<0.0001, Day 22) and slightly prolonged median survival relative to the isotype control (26 vs. 33 days) (FIGS. 4A, B and D). Treatment with a combination of FS122m and anti-mPD1 increased TGI (85.1%) relative to FS122m (P<0.0001, Day 22) and anti-mPD1 (P<0.0001, Day 22) monotherapy (FIG. 4A). Combined FS122m and anti-mPD-1 treatment also prolonged median survival (50 days) (FIG. 4B) and induced complete tumor regression in 3 out of 10 mice, while both FS112 and anti-mPD-1 monotherapy did not achieve complete tumor regression in any of 10 mice (FIG. 4D). No relevant body weight changes were noted across the treatment groups compared with isotype control, demonstrating that all of the treatments were well tolerated (FIG. 4C).

### 3.3 Eph-1424 Mouse Breast Tumor Model

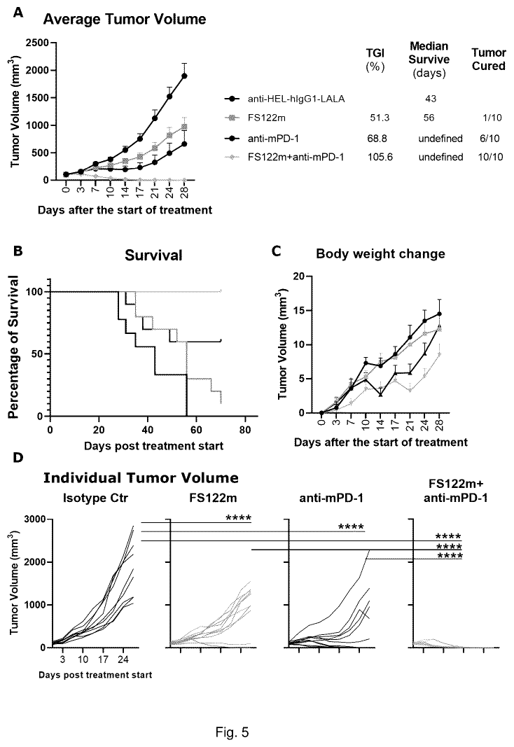

The antitumor efficacy of FS122m in combination with anti-mPD1 was also evaluated in the Eph4-1424 subcutaneous breast tumor model in BALB/c mice. Both anti-mPD1 and FS122m monotherapy displayed significant antitumor effect (TGI=68.8% and 51.3% respectively, P<0.0001) and significantly prolonged median survival relative to the isotype control (43 days vs 56 days vs undefined) (FIGS. 5A, B and D). Treatment with a combination of FS122m and anti-mPD1 increased TGI (105.6%) relative to FS122m (P<0.0001, Day 28) and anti-mPD1 (P<0.0001, Day 28) monotherapy (FIG. 5A). Combined FS122m and anti-mPD-1 treatment also prolonged median survival (FIG. 5B) and induced complete tumor regression in 10 of 10 mice compared with complete tumor regression in only 1 of 10 mice treated with FS122m monotherapy and complete tumor regression in 6 of 10 mice treated with anti-mPD-1 monotherapy (FIG. 5D). No relevant body weight changes were noted across the treatment groups compared to isotype control, suggesting that all of the treatments were well tolerated (FIG. 5C).

Overall, the combined treatment of FS122m and anti-mPD-1 resulted in statistically significantly greater TGI than monotherapy treatment with FS122m or anti-mPD-1. Complete tumor regression was achieved in the majority of mice in two of the three mouse breast tumor models with a combination of FS122m and anti-mPD-1 (FIGS. 3D and 5D). The difference compared with FS122m and anti-PD-1 monotherapy was particularly stark in the E0771 mouse tumor model, where the combination of FS122m with anti-mPD1 resulted in complete tumor regression in 7 out of 9 mice compared with 1 out of 9 mice with anti-mPD1 monotherapy and no mice in the FS122m monotherapy group (FIG. 3D). Complete tumor regression was also achieved in some of the mice in the JC mouse tumor model as a result of combined FS122m and anti-mPD-1 therapy, while both FS112 and anti-mPD-1 monotherapy did not result in complete tumor regression in any of the mice treated (FIG. 4D). In all three mouse tumor models, combination therapy produced prolonged median survival compared with monotherapy (FIGS. 3B, 4B and 5B).

### SEQUENCE LISTING

### Heavy Chain Annotations

i. In amino acid sequences of the heavy chain of mAb², the variable domain is shown in italics, CDRs according to IMGT are shown in bold italics, CDRs according to Kabat are shown in italics and underlined (therefore any overlapping IMGT and Kabat CDR sequences are shown in bold, italics and underlined), CH1 domains are underlined, hinge regions are doubly underlined, CH2 domains are shown in bold (and, where applicable, location of the LALA mutation is shown in bold and underlined), CH3 domains are shown in plain font, and modified regions of CH3 structural loops are underlined (no underlining if loop is unchanged).
ii. In amino acid sequences of variable domains, CDRs according to IMGT are shown in bold and italics, CDRs according to Kabat are shown in italics and underlined (therefore any overlapping IMGT and Kabat CDR sequences are shown in bold, italics and underlined).
iii. CDR amino acid sequences according to both IMGT and Kabat are provided.

### Light Chain Annotations

i. In the amino acid sequence of the light chain of mAb², variable domains are shown in italics, CDRs according to IMGT are shown in bold and italics, and CDRs according to Kabat are shown in italics and underlined (therefore any overlapping IMGT and Kabat CDR sequences are shown in bold, italics and underlined).
ii. In the amino acid sequence of the variable domain, CDRs according to IMGT are shown in bold and italics, and CDRs according to Kabat are shown in italics and underlined (therefore any overlapping IMGT and Kabat CDR sequences are shown in bold, italics and underlined).
iii. CDR amino acid sequences according to both IMGT and Kabat are provided.

Amino acid sequences of FS22-172-003-AA/FS28-256-271 mAb²
SEQ ID NO: 1 Heavy chain AA (without LALA)
EVQLLESGGGLVQPGGSLRLSCAASGFTFTHTYMSWVRQAPGKGLEWVSAI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 2 Heavy chain AA (with LALA)
EVQLLESGGGLVQPGGSLRLSCAASGFTFTHTYMSWVRQAPGKGLEWVSAI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 3 VH domain AA
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSS

| | | |
|---|---|---|
| SEQ ID NO: 4 | HCDR1 (AA) (IMGT) | GFTFTHTY |
| SEQ ID NO: 5 | HCDR1 (AA) (Kabat) | HTYMS |
| SEQ ID NO: 6 | HCDR2 (AA) (IMGT) | ISPTYSTT |
| SEQ ID NO: 7 | HCDR2 (AA) Kabat) | AISPTYSTTNYADSVKG |
| SEQ ID NO: 8 | HCDR3 (AA) (IMGT) | ARYNAYHAALDY |
| SEQ ID NO: 9 | HCDR3 (AA) (Kabat) | YNAYHAALDY |

SEQ ID NO: 10 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 11 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 16 | LCDR3 (AA) (IMGT) | QQTVPYPYT |
| SEQ ID NO: 16 | LCDR3 (AA) (Kabat) | QQTVPYPYT |

Amino acid sequences of FS22-172-003-AA/FS28-024-052 mAb²
SEQ ID NO: 17 Heavy chain AA (without LALA)
EVQLLESGGGLVQPGGSLRLSCAASGFTLSYSSMSWVRQAPGKGLEWVSFI
TPSTGYTHYADSVKGRFTI
SRDNSKNTLYLQMNSLRAEDTAVYYCARRALLFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREP
QVYTLPPSRDELPYIIPPYNQVSLTCLVKGFYPSDIAVEWESNGQPENNYKTTPPVLDSDGSFFLYSKLTV
GADRWLEGNVFSCSVMHEALHNHYTQKSLSLSPG
SEQ ID NO: 18 Heavy chain AA (with LALA)
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALLFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
SEQ ID NO: 19 VH domain AA
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALLFDYWGQGTLVTVSS

| | | |
|---|---|---|
| SEQ ID NO: 20 | HCDR1 (AA) (IMGT) | GFTLSYSS |
| SEQ ID NO: 21 | HCDR1 (AA) (Kabat) | YSSMS |
| SEQ ID NO: 22 | HCDR2 (AA) (IMGT) | ITPSTGYT |
| SEQ ID NO: 23 | HCDR2 (AA) Kabat) | FITPSTGYTHYADSVKG |
| SEQ ID NO: 24 | HCDR3 (AA) (IMGT) | ARRALLFDY |
| SEQ ID NO: 25 | HCDR3 (AA) (Kabat) | RALLFDY |

SEQ ID NO: 26 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 27 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 28 | LCDR3 (AA) (IMGT) | QQASSYPLT |
| SEQ ID NO: 28 | LCDR3 (AA) (Kabat) | QQASSYPLT |

Amino acid sequences of FS22-172-003-AA/FS28-256-021 mAb²
SEQ ID NO: 29 Heavy chain AA (without LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 30 Heavy chain AA (with LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 3 VH domain AA
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSS

| | | |
|---|---|---|
| SEQ ID NO: 4 | HCDR1 (AA) (IMGT) | GFTFTHTY |
| SEQ ID NO: 5 | HCDR1 (AA) (Kabat) | HTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 8 | HCDR3 (AA) (IMGT) | ARYNAYHAALDY |
| SEQ ID NO: 9 | HCDR3 (AA) (Kabat) | YNAYHAALDY |

SEQ ID NO: 32 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 33 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 34 | LCDR3 (AA) (IMGT) | QQHNQYPNT |
| SEQ ID NO: 34 | LCDR3 (AA) (Kabat) | QQHNQYPNT |

Amino acid sequences of FS22-172-003-AA/FS28-256-012 mAb²
SEQ ID NO: 35 Heavy chain AA (without LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 36 Heavy chain AA (with LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 3 VH domain AA
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSS

| | | |
|---|---|---|
| SEQ ID NO: 4 | HCDR1 (AA) (IMGT) | GFTFTHTY |
| SEQ ID NO: 5 | HCDR1 (AA) (Kabat) | HTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 8 | HCDR3 (AA) (IMGT) | ARYNAYHAALDY |
| SEQ ID NO: 9 | HCDR3 (AA) (Kabat) | YNAYHAALDY |

SEQ ID NO: 37 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
SEQ ID NO: 38 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 39 | LCDR3 (AA) (IMGT) | QQSYYYPIT |
| SEQ ID NO: 39 | LCDR3 (AA) (Kabat) | QQSYYYPIT |

Amino acid sequences of FS22-172-003-AA/FS28-256-023 mAb²
SEQ ID NO: 40 Heavy chain AA (without LALA)
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 41 Heavy chain AA (with LALA)
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 42 VH domain AA
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSS

| | | |
|---|---|---|
| SEQ ID NO: 43 | HCDR1 (AA) (IMGT) | GFTFTQTY |
| SEQ ID NO: 44 | HCDR1 (AA) (Kabat) | QTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 45 | HCDR3 (AA) (IMGT) | ARYNAYQIGLDY |
| SEQ ID NO: 46 | HCDR3 (AA) (Kabat) | YNAYQIGLDY |

SEQ ID NO: 32 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 33 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 34 | LCDR3 (AA) (IMGT) | QQHNQYPNT |
| SEQ ID NO: 34 | LCDR3 (AA) (Kabat) | QQHNQYPNT |

Amino acid sequences of FS22-172-003-AA/FS28-256-024 mAb²
SEQ ID NO: 29 Heavy chain AA (without LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 30 Heavy chain AA (with LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 3 VH domain AA
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSS

| | | |
|---|---|---|
| SEQ ID NO: 4 | HCDR1 (AA) (IMGT) | GFTFTHTY |
| SEQ ID NO: 5 | HCDR1 (AA) (Kabat) | HTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 8 | HCDR3 (AA) (IMGT) | ARYNAYHAALDY |
| SEQ ID NO: 9 | HCDR3 (AA) (Kabat) | YNAYHAALDY |

SEQ ID NO: 47 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 48 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 49 | LCDR3 (AA) (IMGT) | QQALGYPHT |
| SEQ ID NO: 49 | LCDR3 (AA) (Kabat) | QQALGYPHT |

Amino acid sequences of FS22-172-003-AA/FS28-256-026 mAb²
SEQ ID NO: 40 Heavy chain AA (without LALA)
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 41 Heavy chain AA (with LALA)
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 42 VH domain AA
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSS

| | | |
|---|---|---|
| SEQ ID NO: 43 | HCDR1 (AA) (IMGT) | GFTFTQTY |
| SEQ ID NO: 44 | HCDR1 (AA) (Kabat) | QTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 45 | HCDR3 (AA) (IMGT) | ARYNAYQIGLDY |
| SEQ ID NO: 46 | HCDR3 (AA) (Kabat) | YNAYQIGLDY |

SEQ ID NO: 47 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 48 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 49 | LCDR3 (AA) (IMGT) | QQALGYPHT |
| SEQ ID NO: 49 | LCDR3 (AA) (Kabat) | QQALGYPHT |

Amino acid sequences of FS22-172-003-AA/FS28-256-027 mAb²
SEQ ID NO: 29 Heavy chain AA (without LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 30 Heavy chain AA (with LALA)
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 3 VH domain AA
EVQLLESGGGLVQPGGSLRLSCAASGFTFTHTYMSWVRQAPGKGLEWVSNI
SPTYSTTNYADSVKGRFTISRDNNKNTLYLQMNSLRAEDTAVYYCARYNAYHAALDYWGQGTLVTVSS

| | | |
|---|---|---|
| SEQ ID NO: 4 | HCDR1 (AA) (IMGT) | GFTFTHTY |
| SEQ ID NO: 5 | HCDR1 (AA) (Kabat) | HTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 8 | HCDR3 (AA) (IMGT) | ARYNAYHAALDY |
| SEQ ID NO: 9 | HCDR3 (AA) (Kabat) | YNAYHAALDY |

SEQ ID NO: 10 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 11 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 16 | LCDR3 (AA) (IMGT) | QQTVPYPYT |
| SEQ ID NO: 16 | LCDR3 (AA) (Kabat) | QQTVPYPYT |

Amino acid sequences of FS22-172-003-AA/FS28-256-001 mAb²
SEQ ID NO: 50 Heavy chain AA (without LALA)
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 51 Heavy chain AA (with LALA)
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
SEQ ID NO: 52 VH domain AA
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS

| | | |
|---|---|---|
| SEQ ID NO: 53 | HCDR1 (AA) (IMGT) | GFTFTETY |
| SEQ ID NO: 54 | HCDR1 (AA) (Kabat) | ETYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 55 | HCDR3 (AA) (IMGT) | ARYNSYQGGLDY |
| SEQ ID NO: 56 | HCDR3 (AA) (Kabat) | YNSYQGGLDY |

SEQ ID NO: 32 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 33 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 34 | LCDR3 (AA) (IMGT) | QQHNQYPNT |
| SEQ ID NO: 34 | LCDR3 (AA) (Kabat) | QQHNQYPNT |

Amino acid sequences of FS22-172-003-AA/FS28-256-005 mAb²
SEQ ID NO: 50 Heavy chain AA (without LALA)
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 51 Heavy chain AA (with LALA)
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
SEQ ID NO: 52 VH domain AA
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS

| | | |
|---|---|---|
| SEQ ID NO: 53 | HCDR1 (AA) (IMGT) | GFTFTETY |
| SEQ ID NO: 54 | HCDR1 (AA) (Kabat) | ETYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 55 | HCDR3 (AA) (IMGT) | ARYNSYQGGLDY |
| SEQ ID NO: 56 | HCDR3 (AA) (Kabat) | YNSYQGGLDY |

SEQ ID NO: 47 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 48 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 49 | LCDR3 (AA) (IMGT) | QQALGYPHT |
| SEQ ID NO: 49 | LCDR3 (AA) (Kabat) | QQALGYPHT |

Amino acid sequences of FS22-172-003-AA/FS28-256-014 mAb²
SEQ ID NO: 57 Heavy chain AA (without LALA)
EVQLLESGGGLVQPGGSLRLSCAASGFTFTDTYMSWVRQAPGKGLEWVSNI
SPTYSTTNYADSVKGRFTISRDNSKNTLYLQMNSLRAEDTAVYYCARYNAYAAGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 58 Heavy chain AA (with LALA)
EVQLLESGGGLVQPGGSLRLSCAASGFTFTDTYMSWVRQAPGKGLEWVSNI
SPTYSTTNYADSVKGRFTISRDNSKNTLYLQMNSLRAEDTAVYYCARYNAYAAGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 59 VH domain AA
EVQLLESGGGLVQPGGSLRLSCAASGFTFTDTYMSWVRQAPGKGLEWVSNI
SPTYSTTNYADSVKGRFTISRDNSKNTLYLQMNSLRAEDTAVYYCARYNAYAAGLDYWGQGTLVTVSS

| | | |
|---|---|---|
| SEQ ID NO: 60 | HCDR1 (AA) (IMGT) | GFTFTDTY |
| SEQ ID NO: 61 | HCDR1 (AA) (Kabat) | DTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 62 | HCDR3 (AA) (IMGT) | ARYNAYAAGLDY |
| SEQ ID NO: 63 | HCDR3 (AA) (Kabat) | YNAYAAGLDY |

SEQ ID NO: 37 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
SEQ ID NO: 38 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 39 | LCDR3 (AA) (IMGT) | QQSYYYPIT |
| SEQ ID NO: 39 | LCDR3 (AA) (Kabat) | QQSYYYPIT |

Amino acid sequences of FS22-172-003-AA/FS28-256-018 mAb²
SEQ ID NO: 40 Heavy chain AA (without LALA)
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
SEQ ID NO: 41 Heavy chain AA (with LALA)
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 42 VH domain AA
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSS

| | | |
|---|---|---|
| SEQ ID NO: 43 | HCDR1 (AA) (IMGT) | GFTFTQTY |
| SEQ ID NO: 44 | HCDR1 (AA) (Kabat) | QTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 45 | HCDR3 (AA) (IMGT) | ARYNAYQIGLDY |
| SEQ ID NO: 46 | HCDR3 (AA) (Kabat) | YNAYQIGLDY |

SEQ ID NO: 37 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
SEQ ID NO: 38 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 39 | LCDR3 (AA) (IMGT) | QQSYYYPIT |
| SEQ ID NO: 39 | LCDR3 (AA) (Kabat) | QQSYYYPIT |

Amino acid sequences of FS22-172-003-AA/FS28-256 mAb²
SEQ ID NO: 64 Heavy chain AA (without LALA)
EVQLLESGGGLVQPGGSLRLSCAASGFTFTNTYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
SEQ ID NO: 65 Heavy chain AA (with LALA)
EVQLLESGGGLVQPGGSLRLSCAASGFTFTNTYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
SEQ ID NO: 66 VH domain AA
EVQLLESGGGLVQPGGSLRLSCAASGFTFTNTYMSWVRQAPGKGLEWVSNI
SPTYSTTNYADSVKGRFTISRDNSKNTLYLQMNSLRAEDTAVYYCARYNSYQGGLDYWGQGTLVTVSS

| | | |
|---|---|---|
| SEQ ID NO: 67 | HCDR1 (AA) (IMGT) | GFTFTNTY |
| SEQ ID NO: 68 | HCDR1 (AA) (Kabat) | NTYMS |
| SEQ ID NO: 6 | HCDR2 (AA) (IMGT) | ISPTYSTT |
| SEQ ID NO: 31 | HCDR2 (AA) Kabat) | NISPTYSTTNYADSVKG |
| SEQ ID NO: 55 | HCDR3 (AA) (IMGT) | ARYNSYQGGLDY |
| SEQ ID NO: 56 | HCDR3 (AA) (Kabat) | YNSYQGGLDY |

SEQ ID NO: 37 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
SEQ ID NO: 38 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 39 | LCDR3 (AA) (IMGT) | QQSYYYPIT |
| SEQ ID NO: 39 | LCDR3 (AA) (Kabat) | QQSYYYPIT |

Amino acid sequences of FS22-172-003-AA/FS28-024-051 mAb^{²}
SEQ ID NO: 69 Heavy chain AA (without LALA)
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALIFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
SEQ ID NO: 70 Heavy chain AA (with LALA)
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALIFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
SEQ ID NO: 71 VH domain AA
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALIFDYWGQGTLVTVSS

| | | |
|---|---|---|
| SEQ ID NO: 21 | HCDR1 (AA) (IMGT) | GFTLSYSS |
| SEQ ID NO: 22 | HCDR1 (AA) (Kabat) | YSSMS |
| SEQ ID NO: 23 | HCDR2 (AA) (IMGT) | ITPSTGYT |
| SEQ ID NO: 24 | HCDR2 (AA) Kabat) | FITPSTGYTHYADSVKG |
| SEQ ID NO: 72 | HCDR3 (AA) (IMGT) | ARRALIFDY |
| SEQ ID NO: 73 | HCDR3 (AA) (Kabat) | RALIFDY |

SEQ ID NO: 26 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 27 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 28 | LCDR3 (AA) (IMGT) | QQASSYPLT |
| SEQ ID NO: 28 | LCDR3 (AA) (Kabat) | QQASSYPLT |

Amino acid sequences of FS22-172-003-AA/FS28-024-053 mAb²
SEQ ID NO: 74 Heavy chain AA (without LALA)
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALVFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
SEQ ID NO: 75 Heavy chain AA (with LALA)
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALVFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
SEQ ID NO: 76 VH domain AA
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALVFDYWGQGTLVTVSS

| | | |
|---|---|---|
| SEQ ID NO: 21 | HCDR1 (AA) (IMGT) | GFTLSYSS |
| SEQ ID NO: 22 | HCDR1 (AA) (Kabat) | YSSMS |
| SEQ ID NO: 23 | HCDR2 (AA) (IMGT) | ITPSTGYT |
| SEQ ID NO: 24 | HCDR2 (AA) Kabat) | FITPSTGYTHYADSVKG |
| SEQ ID NO: 77 | HCDR3 (AA) (IMGT) | ARRALVFDY |
| SEQ ID NO: 78 | HCDR3 (AA) (Kabat) | RALVFDY |

SEQ ID NO: 26 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 27 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 28 | LCDR3 (AA) (IMGT) | QQASSYPLT |
| SEQ ID NO: 28 | LCDR3 (AA) (Kabat) | QQASSYPLT |

Amino acid sequences of FS22-172-003-AA/FS28-024 mAb²
SEQ ID NO: 79 Heavy chain AA (without LALA)
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALTFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
SEQ ID NO: 80 Heavy chain AA (with LALA)
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALTFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
SEQ ID NO: 81 VH domain AA
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALTFDYWGQGTLVTVSS

| | | |
|---|---|---|
| SEQ ID NO: 21 | HCDR1 (AA) (IMGT) | GFTLSYSS |
| SEQ ID NO: 22 | HCDR1 (AA) (Kabat) | YSSMS |
| SEQ ID NO: 23 | HCDR2 (AA) (IMGT) | ITPSTGYT |
| SEQ ID NO: 24 | HCDR2 (AA) Kabat) | FITPSTGYTHYADSVKG |
| SEQ ID NO: 82 | HCDR3 (AA) (IMGT) | ARRALTFDY |
| SEQ ID NO: 83 | HCDR3 (AA) (Kabat) | RALTFDY |

SEQ ID NO: 26 Light chain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 27 VL domain AA
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK

| | | |
|---|---|---|
| SEQ ID NO: 12 | LCDR1 (AA) (IMGT) | QSVSSSY |
| SEQ ID NO: 13 | LCDR1 (AA) (Kabat) | RASQSVSSSYLA |
| SEQ ID NO: 14 | LCDR2 (AA) (IMGT) | GAS |
| SEQ ID NO: 15 | LCDR2 (AA) (Kabat) | GASSRAT |
| SEQ ID NO: 28 | LCDR3 (AA) (IMGT) | QQASSYPLT |
| SEQ ID NO: 28 | LCDR3 (AA) (Kabat) | QQASSYPLT |

Amino acid sequences of FS122m (surrogate anti-mMSLN mCD137 Fcab G1-AA):
SEQ ID NO: 84 Heavy chain AA (with LALA)
EVQLLESGGGLVQPGGSLRLSCAASGFTFSTYFMVWVRQAPGKGLEWVSMIS
PKSSNTYYADSVKGRFTISRDNSKNTLYLQMNSLRAEDTAVYYCARYHISPRFDYWGQGTLVTVSSAST
KGPSVFPLAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEAAGGPSVFLFPPKPKDTLMISRTPEV
TCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDEPYWSYVSLTCLVKGFYPSDIAVEWESNGQPENNYKTTPPV
LDSDGSFFLYSKLTVMNYRWELGNVFSCSVMHEALHNHYTQKSLSLSPGK
SEQ ID NO: 85 Light chain AA
EIVLTQSPGTLSLSPGERATLSCRASQSVSSSYLAWYQQKPGQAPRLLIYGASS
RATGIPDRFSGSGSGTDFTLTISRLEPEDFAVYYCQQPFPFSFTFGQGTKVEIKRTVAAPSVFIFPPSDEQ
LKSGTASVVCLLNNFYPREAKVQWKVDNALQSGNSQESVTEQDSKDSTYSLSSTLTLSKADYEKHKVYA
CEVTHQGLSSPVTKSFNRGEC
Amino acid sequences of CH3 domain and amino acid sequence of modified regions
of CH3 AB and EF structural loops of all FS22-172-003 Fcab-containing mAb²
clones and the FS22-172-003 Fcab
SEQ ID NO: 86 CH3 domain
GQPREPQVYTLPPSRDELPYIIPPYNQVSLTCLVKGFYPSDIAVEWESNGQP
ENNYKTTPPVLDSDGSFFLYSKLTVGADRWLEGNVFSCSVMHEALHNHYTQKSLSLSPG

| | | |
|---|---|---|
| SEQ ID NO: 87 | AB Loop | PYIIPPY |
| SEQ ID NO: 88 | EF Loop | GADRWLE |
| SEQ ID NO: 89 | CD Loop | SNGQPENNY |

Amino acid sequences of CH2 domain containing LALA mutation, PA mutation or
LALA-PA mutation (mutations in bold and underlined)
SEQ ID NO: 90 CH2 (LALA)
APEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
SEQ ID NO: 91 CH2 (PA)
APELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALAAPIEKTISKAK
SEQ ID NO: 92 CH2 (LALA-PA)
APEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALAAPIEKTISKAK
Amino acid sequence of pembrolizumab
SEQ ID NO: 93 Heavy chain AA
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCARRDYRFDMGFDYWGQGTTVTV
SSASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK
SEQ ID NO: 94 Light chain AA
EIVLTQSPATLSLSPGERATLSCRASKGVSTSGYSYLHWYQQKPGQAPRLLI
YLASYLESGVPARFSGSGSGTDFTLTISSLEPEDFAVYYCQHSRDLPLTFGGGTKVEIKRTVAAPSVFIFP
PSDEQLKSGTASVVCLLNNFYPREAKVQWKVDNALQSGNSQESVTEQDSKDSTYSLSSTLTLSKADYEK
HKVYACEVTHQGLSSPVTKSFNRGEC

### REFERENCES

A number of publications are cited above in order to more fully describe and disclose the invention and the state of the art to which the invention pertains. Full citations for these references are provided below. The entirety of each of these references is incorporated herein.
1 de Miguel, M. & Calvo, E. Clinical Challenges of Immune Checkpoint Inhibitors. Cancer Cell 38, 326-333, doi:10.1016/j.ccell.2020.07.004 (2020).
2 Bartkowiak, T. & Curran, M. A. 4-1BB Agonists: Multi-Potent Potentiators of Tumor Immunity. Front Oncol 5, 117, doi:10.3389/fonc.2015.00117 (2015).
3 Compte, M. et al. A tumor-targeted trimeric 4-1BB-agonistic antibody induces potent anti-tumor immunity without systemic toxicity. Nat Commun 9, 4809, doi:10.1038/s41467-018-07195-w (2018).
4 Kataoka, K. et al. Integrated molecular analysis of adult T cell leukemia/lymphoma. Nat Genet 47, 1304-1315, doi:10.1038/ng.3415 (2015).
5 Sanmamed, M. F. et al. Agonists of Co-stimulation in Cancer Immunotherapy Directed Against CD137, OX40, GITR, CD27, CD28, and ICOS. Semin Oncol 42, 640-655, doi:10.1053/j.seminoncol.2015.05.014 (2015).
6 Yonezawa, A., Chester, C., Rajasekaran, N. & Kohrt, H. E. Harnessing the innate immune system to treat cancer: enhancement of antibody-dependent cellular cytotoxicity with anti-CD137 Ab. Chin Clin Oncol 5, 5, doi:10.3978/j.issn.2304-3865.2016.02.05 (2016).
7 Yonezawa, A., Dutt, S., Chester, C., Kim, J. & Kohrt, H. E. Boosting Cancer Immunotherapy with Anti-CD137 Antibody Therapy. Clin Cancer Res 21, 3113-3120, doi:10.1158/1078-0432.CCR-15-0263 (2015).
8 Melero, I., Johnston, J. V., Shufford, W. W., Mittler, R. S. & Chen, L. NK1.1 cells express 4-1BB (CDw137) costimulatory molecule and are required for tumor immunity elicited by anti-4-1BB monoclonal antibodies. Cell Immunol 190, 167-172, doi:10.1006/cimm. 1998.1396 (1998).
9 Vinay, D. S. & Kwon, B. S. Role of 4-1BB in immune responses. Semin Immunol 10, 481-489, doi:10.1006/smim. 1998.0157 (1998).
10 Cannons, J. L., Hoeflich, K. P., Woodgett, J. R. & Watts, T. H. Role of the stress kinase pathway in signaling via the T cell costimulatory receptor 4-1BB. J Immunol 163, 2990-2998 (1999).
11 Sica, G. & Chen, L. Biochemical and immunological characteristics of 4-1BB (CD137) receptor and ligand and potential applications in cancer therapy. Arch Immunol Ther Exp (Warsz) 47, 275-279 (1999).
12 Lee, H. W. et al. 4-1BB promotes the survival of CD8+T lymphocytes by increasing expression of Bcl-xL and Bfl-1. J Immunol 169, 4882-4888, doi:10.4049/jimmunol. 169.9.4882 (2002).
13 Lee, H. W., Nam, K. O., Park, S. J. & Kwon, B. S. 4-1BB enhances CD8+ T cell expansion by regulating cell cycle progression through changes in expression of cyclins D and E and cyclin-dependent kinase inhibitor p27kip1. Eur J Immunol 33, 2133-2141, doi:10.1002/eji.200323996 (2003).
14 Chacon, J. A. et al. Co-stimulation through 4-1BB/CD137 improves the expansion and function of CD8(+) melanoma tumor-infiltrating lymphocytes for adoptive T-cell therapy. PLoS One 8, e60031, doi:10.1371/journal.pone.0060031 (2013).
15 Fisher, T. S. et al. Targeting of 4-1BB by monoclonal antibody PF-05082566 enhances T-cell function and promotes anti-tumor activity. Cancer Immunol Immunother 61, 1721-1733, doi:10.1007/s00262-012-1237-1 (2012).
16 Claus, C. et al. Tumor-targeted 4-1BB agonists for combination with T cell bispecific antibodies as off-the-shelf therapy. Sci Transl Med 11, doi:10.1126/scitranslmed.aav5989 (2019).
17 Qi, X. et al. Optimization of 4-1BB antibody for cancer immunotherapy by balancing agonistic strength with FcgammaR affinity. Nat Commun 10, 2141, doi:10.1038/s41467-019-10088-1 (2019).
18 Azpilikueta, A. et al. Successful Immunotherapy against a Transplantable Mouse Squamous Lung Carcinoma with Anti-PD-1 and Anti-CD137 Monoclonal Antibodies. J Thorac Oncol 11, 524-536, doi:10.1016/j.jtho.2016.01.013 (2016).
19 Narazaki, H., Zhu, Y., Luo, L., Zhu, G. & Chen, L. CD137 agonist antibody prevents cancer recurrence: contribution of CD137 on both hematopoietic and nonhematopoietic cells. Blood 115, 1941-1948, doi:10.1182/blood-2008-12-192591 (2010).
20 Wilcox, R. A. et al. Provision of antigen and CD137 signaling breaks immunological ignorance, promoting regression of poorly immunogenic tumors. J Clin Invest 109, 651-659, doi:10.1172/JCI14184 (2002).
21 Weigelin, B. et al. Focusing and sustaining the antitumor CTL effector killer response by agonist anti-CD137 mAb. Proc Natl Acad Sci USA 112, 7551-7556, doi:10.1073/pnas. 1506357112 (2015).
22 Segal, N. H. et al. Results from an Integrated Safety Analysis of Urelumab, an Agonist Anti-CD137 Monoclonal Antibody. Clin Cancer Res 23, 1929-1936, doi:10.1158/1078-0432.CCR-16-1272 (2017).
23 Timmerman, J. et al. Urelumab alone or in combination with rituximab in patients with relapsed or refractory B-cell lymphoma. Am J Hematol 95, 510-520, doi:10.1002/ajh.25757 (2020).
24 Chin, S. M. et al. Structure of the 4-1BB/4-1BBL complex and distinct binding and functional properties of utomilumab and urelumab. Nat Commun 9, 4679, doi:10.1038/s41467-018-07136-7 (2018).
25 Eskiocak, U. et al. Differentiated agonistic antibody targeting CD137 eradicates large tumors without hepatotoxicity. JCI Insight 5, doi:10.1172/jci.insight. 133647 (2020).
26 Trub, M. et al. Fibroblast activation protein-targeted-4-1BB ligand agonist amplifies effector functions of intratumoral T cells in human cancer. J Immunother Cancer 8, doi:10.1136/jitc-2019-000238 (2020).
27 Mardiana, S. et al. A Multifunctional Role for Adjuvant Anti-4-1BB Therapy in Augmenting Antitumor Response by Chimeric Antigen Receptor T Cells. Cancer Res 77, 1296-1309, doi:10.1158/0008-5472.CAN-16-1831 (2017).
28 Ordóñez, N. G. Application of mesothelin immunostaining in tumor diagnosis. Am J Surg Pathol 27, 1418-1428, doi:10.1097/00000478-200311000-00003 (2003).
29 Chang, K. & Pastan, I. Molecular cloning of mesothelin, a differentiation antigen present on mesothelium, mesotheliomas, and ovarian cancers. Proc Natl Acad Sci USA 93, 136-140, doi:10.1073/pnas.93.1.136 (1996).
30 Miettinen, M. & Sarlomo-Rikala, M. Expression of calretinin, thrombomodulin, keratin 5, and mesothelin in lung carcinomas of different types: an immunohistochemical analysis of 596 tumors in comparison with epithelioid mesotheliomas of the pleura. Am J Surg Pathol 27, 150-158, doi:10.1097/00000478-200302000-00002 (2003).
31 Hassan, R. et al. Anti-tumor activity of K1-LysPE38QQR, an immunotoxin targeting mesothelin, a cell-surface antigen overexpressed in ovarian cancer and malignant mesothelioma. J Immunother 23, 473-479, doi:10.1097/00002371-200007000-00011 (2000).
32 Hassan, R. et al. Phase 1 study of the immunotoxin LMB-100 in patients with mesothelioma and other solid tumors expressing mesothelin. Cancer 126, 4936-4947, doi:10.1002/cncr.33145 (2020).
33 Hassan, R. et al. Clinical Response of Live-Attenuated, Listeria monocytogenes Expressing Mesothelin (CRS-207) with Chemotherapy in Patients with Malignant Pleural Mesothelioma. Clin Cancer Res 25, 5787-5798, doi:10.1158/1078-0432.CCR-19-0070 (2019).
34 Hassan, R. et al. First-in-Human, Multicenter, Phase I Dose-Escalation and Expansion Study of Anti-Mesothelin Antibody-Drug Conjugate Anetumab Ravtansine in Advanced or Metastatic Solid Tumors. J Clin Oncol 38, 1824-1835, doi:10.1200/JCO.19.02085 (2020).
35 Hassan, R. et al. Mesothelin Immunotherapy for Cancer: Ready for Prime Time? J Clin Oncol 34, 4171-4179, doi:10.1200/JCO.2016.68.3672 (2016).
36 Hassan, R. et al. Inhibition of mesothelin-CA-125 interaction in patients with mesothelioma by the anti-mesothelin monoclonal antibody MORAb-009: Implications for cancer therapy. Lung Cancer 68, 455-459, doi:10.1016/j.lungcan.2009.07.016 (2010).
37 Kreitman, R. J., Hassan, R., Fitzgerald, D. J. & Pastan, I. Phase I trial of continuous infusion anti-mesothelin recombinant immunotoxin SS1P. Clin Cancer Res 15, 5274-5279, doi:10.1158/1078-0432.CCR-09-0062 (2009).
38 Hassan, R. et al. Phase I study of SS1P, a recombinant anti-mesothelin immunotoxin given as a bolus I.V. infusion to patients with mesothelin-expressing mesothelioma, ovarian, and pancreatic cancers. Clin Cancer Res 13, 5144-5149, doi:10.1158/1078-0432.CCR-07-0869 (2007).
39 Ho, M. et al. Mesothelin expression in human lung cancer. Clin Cancer Res 13, 1571-1575, doi:10.1158/1078-0432.CCR-06-2161 (2007).
40 Kelly, R. J., Sharon, E., Pastan, I. & Hassan, R. Mesothelin-targeted agents in clinical trials and in preclinical development. Mol Cancer Ther 11, 517-525, doi:10.1158/1535-7163.MCT-11-0454 (2012).
41 Twomey, J. D. & Zhang, B. Cancer Immunotherapy Update: FDA-Approved Checkpoint Inhibitors and Companion Diagnostics. Aaps j 23, 39, doi:10.1208/s12248-021-00574-0 (2021).
42 Mullard, A. LAG3 pushes immuno-oncology's leading edge. Nat Rev Drug Discov 21, 167-169, doi:10.1038/d41573-022-00036-y (2022).
43 Vezys, V. et al. 4-1BB signaling synergizes with programmed death ligand 1 blockade to augment CD8 T cell responses during chronic viral infection. J Immunol 187, 1634-1642, doi:10.4049/jimmunol.1100077 (2011).
44 Wang, S. & Chen, L. Immunobiology of cancer therapies targeting CD137 and B7-H1/PD-1 cosignal pathways. Curr Top Microbiol Immunol 344, 245-267, doi:10.1007/82_2010_81 (2011).
45 Tallarida RJ. Quantitative methods for assessing drug synergism. Genes Cancer 2(11):1003-8. doi:10.1177/1947601912440575 (2011).

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An antibody molecule that binds MSLN and CD137.

2. A method of treating cancer in an individual, the method comprising administering to the individual a therapeutically effective amount of an antibody molecule that binds MSLN and CD137 and a PD-1/PD-L1 inhibitor.

3. A kit comprising
(a) an antibody molecule that binds MSLN and CD137 and a pharmaceutically acceptable excipient; and
(b) a PD-1/PD-L1 inhibitor and a pharmaceutically acceptable excipient.

4. The method according to claim 2, wherein the antibody molecule that binds MSLN and CD137 comprises
(a) a complementary determining region (CDR)-based antigen-binding site for MSLN; and
(b) a CD137 antigen-binding site located in a CH3 domain of the antibody molecule.

5. The method according to claim 2, wherein the CDR-based antigen-binding site for MSLN comprises CDRs 1-6 set forth in:
(i) SEQ ID NOs 4, 6, 8, 12, 14, and 16, respectively [FS28-256-271];
(ii) SEQ ID NOs 20, 22, 24, 12, 14 and 28, respectively [FS28-024-052];
(iii) SEQ ID NOs 4, 6, 8, 12, 14 and 34, respectively [FS28-256-021];
(iv) SEQ ID NOs 4, 6, 8, 12, 14, and 39, respectively [FS28-256-012];
(v) SEQ ID NOs 43, 6, 45, 12, 14 and 34, respectively [FS28-256-023];
(vi) SEQ ID NOs 4, 6, 8, 12, 14 and 49, respectively [FS28-256-024];
(vii) SEQ ID NOs 43, 6, 45, 12, 14 and 49, respectively [FS28-256-026];
(viii) SEQ ID NOs 4, 6, 8, 12, 14 and 16, respectively [FS28-256-027];
(ix) SEQ ID NOs 53, 6, 55, 12, 14 and 34, respectively [FS28-256-001];
(x) SEQ ID NOs 53, 6, 55, 12, 14 and 49, respectively [FS28-256-005];
(xi) SEQ ID NOs 60, 6, 62, 12, 14 and 39, respectively [FS28-256-014];
(xii) SEQ ID NOs 43, 6, 45, 12, 14 and 39, respectively [FS28-256-018];
(xiii) SEQ ID NOs 67, 6, 55, 12, 14 and 39, respectively [FS28-256];
(xiv) SEQ ID NOs 21, 23, 72, 12, 14 and 28, respectively [FS28-024-051];
(XV) SEQ ID NOs 21, 23, 77, 12, 14 and 28, respectively [FS28-024-053]; or
(xvi) SEQ ID NOs 21, 23, 82, 12, 14 and 28, respectively [FS28-024]; and
wherein the CD137 antigen-binding site comprises a first sequence and a second sequence located in the AB and EF structural loops of the CH3 domain, respectively, wherein the first and second sequence have the sequence set forth in SEQ ID NOs 87 and 88 [FS22-172-003], respectively.

6. The method according to claim 2, wherein
(i) the first sequence is located between positions 14 and 17 of the CH3 domain of the antibody molecule; and/or
(ii) wherein the second sequence is located between 91 and 99 of the CH3 domain of the antibody molecule; and
wherein the amino acid residue numbering is according to the IMGT numbering scheme.

7. The method according to claim 2, wherein the antibody molecule comprises the CH3 domain sequence set forth in SEQ ID NO: 86 [FS22-172-003].

8. The method according to claim 2, wherein the antibody molecule comprises the heavy chain and light chain of antibody:
(i) FS22-172-003-AA/FS28-256-271 set forth in SEQ ID NOs 2 and 10, respectively;
(ii) FS22-172-003-AA/FS28-024-052 set forth in SEQ ID NOs 18 and 26, respectively;
(iii) FS22-172-003-AA/FS28-256-021 set forth in SEQ ID NOs 30 and 32, respectively;
(iv) FS22-172-003-AA/FS28-256-012 set forth in SEQ ID NOs 36 and 37, respectively;
(v) FS22-172-003-AA/FS28-256-023 set forth in SEQ ID NOs 41 and 32, respectively;
(vi) FS22-172-003-AA/FS28-256-024 set forth in SEQ ID NOs 30 and 47, respectively;
(vii) FS22-172-003-AA/FS28-256-026 set forth in SEQ ID NOs 41 and 47, respectively;
(viii) FS22-172-003-AA/FS28-256-027 set forth in SEQ ID NOs 30 and 10, respectively;
(ix) FS22-172-003-AA/FS28-256-001 set forth in SEQ ID NOs 51 and 32, respectively;
(x) FS22-172-003-AA/FS28-256-005 set forth in SEQ ID NOs 51 and 47, respectively;
(xi) FS22-172-003-AA/FS28-256-014 set forth in SEQ ID NOs 58 and 37, respectively;
(xii) FS22-172-003-AA/FS28-256-018 set forth in SEQ ID NOs 41 and 37, respectively;
(xiii) FS22-172-003-AA/FS28-256 set forth in SEQ ID NOs 65 and 37, respectively;
(xiv) FS22-172-003-AA/FS28-024-051 set forth in SEQ ID NOs 70 and 26, respectively;
(xv) FS22-172-003-AA/FS28-024-053 set forth in SEQ ID NOs 75 and 26, respectively;
or
(xvi) FS22-172-003-AA/FS28-024 set forth in SEQ ID NOs 80 and 26, respectively.

9. The method according to claim 8, wherein the antibody molecule comprises the heavy chain sequence set forth in SEQ ID NO: 2 and the light chain sequence set forth in SEQ ID NO: 10 [FS22-172-003-AA/FS28-256-271].

10. The method according to claim 2, wherein the PD-1/PD-L1 inhibitor is an antibody that binds PD-1 or PD-L1.

11. The method according to claim 10, wherein the antibody molecule that binds PD-1 is selected from the group consisting of nivolumab, pembrolizumab, and cemiplimab.

12. The method according to claim 10, wherein the antibody molecule that binds PD-L1 is selected from the group consisting of avelumab, atezolizumab, and durvalumab.

13. The method according to claim 2, wherein the cancer is selected from the group consisting of: ovarian cancer, pancreatic adenocarcinoma, mesothelioma, cervical carcinoma, and non-small cell lung carcinoma.

14. The method according to claim 2, wherein treatment with the antibody molecule that binds MSLN and CD137 and the PD-1/PD-L1 inhibitor results in a greater anti-tumor effect than the combined anti-tumor effect that results from treatment of patients with either the antibody molecule that binds MSLN and CD137, or the PD-1/PD-L1 inhibitor, alone.

15. The method according to claim 14, wherein the anti-tumor effect is tumor growth inhibition, tumor volume reduction, increase in median survival, and/or increase in the percentage of patients experiencing complete tumor regression.

16. The method according to claim 2, wherein the antibody molecule that binds MSLN and CD137, and the PD-1/PD-L1 inhibitor, are administered to the patient concomitantly or sequentially.

17. The method according to claim 2, wherein the method comprises determining whether the cancer expresses MSLN and treating the individual if the cancer expresses MSLN.

18. The antibody molecule according to claim 1, wherein the antibody molecule that binds MSLN and CD137 comprises
(a) a complementary determining region (CDR)-based antigen-binding site for MSLN; and
(b) a CD137 antigen-binding site located in a CH3 domain of the antibody molecule.

19. The antibody molecule according to claim 1, wherein the CDR-based antigen-binding site for MSLN comprises CDRs 1-6 set forth in:
(i) SEQ ID NOs 4, 6, 8, 12, 14, and 16, respectively [FS28-256-271];
(ii) SEQ ID NOs 20, 22, 24, 12, 14 and 28, respectively [FS28-024-052];
(iii) SEQ ID NOs 4, 6, 8, 12, 14 and 34, respectively [FS28-256-021];
(iv) SEQ ID NOs 4, 6, 8, 12, 14, and 39, respectively [FS28-256-012];
(v) SEQ ID NOs 43, 6, 45, 12, 14 and 34, respectively [FS28-256-023];
(vi) SEQ ID NOs 4, 6, 8, 12, 14 and 49, respectively [FS28-256-024];
(vii) SEQ ID NOs 43, 6, 45, 12, 14 and 49, respectively [FS28-256-026];
(viii) SEQ ID NOs 4, 6, 8, 12, 14 and 16, respectively [FS28-256-027];
(ix) SEQ ID NOs 53, 6, 55, 12, 14 and 34, respectively [FS28-256-001];
(x) SEQ ID NOs 53, 6, 55, 12, 14 and 49, respectively [FS28-256-005];
(xi) SEQ ID NOs 60, 6, 62, 12, 14 and 39, respectively [FS28-256-014];
(xii) SEQ ID NOs 43, 6, 45, 12, 14 and 39, respectively [FS28-256-018];
(xiii) SEQ ID NOs 67, 6, 55, 12, 14 and 39, respectively [FS28-256];
(xiv) SEQ ID NOs 21, 23, 72, 12, 14 and 28, respectively [FS28-024-051];
(XV) SEQ ID NOs 21, 23, 77, 12, 14 and 28, respectively [FS28-024-053]; or
(xvi) SEQ ID NOs 21, 23, 82, 12, 14 and 28, respectively [FS28-024]; and
wherein the CD137 antigen-binding site comprises a first sequence and a second sequence located in the AB and EF structural loops of the CH3 domain, respectively, wherein the first and second sequence have the sequence set forth in SEQ ID NOs 87 and 88 [FS22-172-003], respectively.

20. The antibody molecule according to claim 1, wherein the antibody molecule comprises the heavy chain and light chain of antibody:
(i) FS22-172-003-AA/FS28-256-271 set forth in SEQ ID NOs 2 and 10, respectively;
(ii) FS22-172-003-AA/FS28-024-052 set forth in SEQ ID NOs 18 and 26, respectively;
(iii) FS22-172-003-AA/FS28-256-021 set forth in SEQ ID NOs 30 and 32, respectively;
(iv) FS22-172-003-AA/FS28-256-012 set forth in SEQ ID NOs 36 and 37, respectively;
(v) FS22-172-003-AA/FS28-256-023 set forth in SEQ ID NOs 41 and 32, respectively;
(vi) FS22-172-003-AA/FS28-256-024 set forth in SEQ ID NOs 30 and 47, respectively;
(vii) FS22-172-003-AA/FS28-256-026 set forth in SEQ ID NOs 41 and 47, respectively;
(viii) FS22-172-003-AA/FS28-256-027 set forth in SEQ ID NOs 30 and 10, respectively;
(ix) FS22-172-003-AA/FS28-256-001 set forth in SEQ ID NOs 51 and 32, respectively;
(x) FS22-172-003-AA/FS28-256-005 set forth in SEQ ID NOs 51 and 47, respectively;
(xi) FS22-172-003-AA/FS28-256-014 set forth in SEQ ID NOs 58 and 37, respectively;
(xii) FS22-172-003-AA/FS28-256-018 set forth in SEQ ID NOs 41 and 37, respectively;
(xiii) FS22-172-003-AA/FS28-256 set forth in SEQ ID NOs 65 and 37, respectively;
(xiv) FS22-172-003-AA/FS28-024-051 set forth in SEQ ID NOs 70 and 26, respectively;
(xv) FS22-172-003-AA/FS28-024-053 set forth in SEQ ID NOs 75 and 26, respectively;
or
(xvi) FS22-172-003-AA/FS28-024 set forth in SEQ ID NOs 80 and 26, respectively.
